(19) United States
(12) Patent Application Publication
(10) Pub. No.: US 2023/0140196 A1
(43) Pub. Date: May 4, 2023

(54) VIRAL VECTOR DOSING PROTOCOLS

(71) Applicant: Selecta Biosciences, Inc., Watertown, MA (US)

(72) Inventor: Takashi Kei Kishimoto, Lexington, MA (US)

(21) Appl. No.: 17/964,523

(22) Filed: Oct. 12, 2022

Related U.S. Application Data
(60) Provisional application No. 63/254,760, filed on Oct. 12, 2021.

(51) Int. Cl. A61K 47/69; C12N 15/86; A61K 31/436

(52) U.S. Cl. CPC A61K 47/6937; A61K 31/436; C12N 15/86; A61K 48/00; C12N 2750/14143; C12N 2750/14171

(57) ABSTRACT

Disclosed, at least in part, are dosings of viral vectors concomitantly with synthetic nanocarriers attached to an immunosuppressant, in combination with dosings of the synthetic nanocarriers attached to an immunosuppressant without a viral vector or further dosings of the synthetic nanocarriers attached to an immunosuppressant concomitantly with doses of the viral vector, and related compositions that provide reduced humoral immune responses and/or increased or durable transgene or nucleic acid material expression.

### RELATED APPLICATIONS

This application claims the benefit of priority under 35 U.S.C. § 119(e) of U.S. Provisional Application Serial No. 63/254,760, filed on Oct. 12, 2021, the entire contents of which are incorporated herein by reference.

### FIELD OF THE INVENTION

This invention relates to doses of viral vectors administered concomitantly with synthetic nanocarriers attached to an immunosuppressant, and related compositions, wherein the doses of the viral vectors may be higher, such as at least 1e¹³ or 2e¹³. In some embodiments, the doses of the viral vectors may be lower, such as lower but at least ⅒. The methods and compositions provided herein may provide reduced humoral immune responses and/or increased or durable transgene or nucleic acid material expression. In one embodiment of any one of the methods provided herein, the synthetic nanocarriers comprising an immunosuppressant is administered monthly, such as concomitantly with a viral vector.

This invention also relates to dosings of viral vectors concomitantly with synthetic nanocarriers attached to an immunosuppressant, in combination with dosings of the synthetic nanocarriers attached to an immunosuppressant without a viral vector or dosings of the synthetic nanocarriers attached to an immunosuppressant concomitantly with lower doses of the viral vector, and related compositions that provide reduced humoral immune responses and/or increased or durable transgene or nucleic acid material expression.

### SUMMARY OF THE INVENTION

In one aspect, a method comprising: (1) a first dosing that comprises concomitantly administering (a) a viral vector, such as an AAV vector, that is not attached to any synthetic nanocarriers, and (b) synthetic nanocarriers that are attached to an immunosuppressant, such as rapamycin, and that comprise no viral vector antigen-presenting cell (APC) presentable antigens of the viral vector; (2) a second dosing that comprises concomitantly administering (c) the synthetic nanocarriers that are attached to an immunosuppressant and that comprise no viral vector APC antigens of the viral vector and the viral vector; and (3) administering the first and second dosings to a subject according to an administration schedule that reduces an undesired humoral immune response to the viral vector and/or increases transgene or nucleic acid material expression or provides durable transgene or nucleic acid material expression, such as for at least one month from the first or second dosing, wherein the viral vector of the first and second dosings is at a higher dose such as at at least 1e¹³ or 2e¹³ vg/kg, is provided.

In one embodiment, the method further comprises: (4) a third dosing that comprises concomitantly administering (d) the synthetic nanocarriers that are attached to an immunosuppressant and that comprise no viral vector APC antigens of the viral vector and the viral vector, wherein the viral vector is also at a higher dose such as at at least 1e¹³ or 2e¹³ vg/kg; and (5) administering the third dosing to a subject also according to an administration schedule that reduces an undesired humoral immune response to the viral vector and/or increases transgene or nucleic acid material expression or provides durable transgene or nucleic acid material expression, such as for at least one month from the third dosing.

In another embodiment, the method further comprises (6) determining the administration schedule for the first and second dosings or first, second and third dosings that reduces an undesired humoral immune response to the viral vector and/ or increases transgene or nucleic acid material expression or provides durable transgene or nucleic acid material expression, such as for at least one month from each first dosing.

In one embodiment of any one of the methods or compositions provided herein, the higher dose is a therapeutically effective dose for a human.

In an aspect, a method comprising (1) a first dosing that comprises concomitantly administering (a) a viral vector, such as an AAV vector, that is not attached to any synthetic nanocarriers, and (b) synthetic nanocarriers that are attached to an immunosuppressant, such as rapamycin, and that comprise no viral vector antigen-presenting cell (APC) presentable antigens of the viral vector; (2) a second dosing that comprises administering (c) the synthetic nanocarriers that are attached to an immunosuppressant and that comprise no viral vector APC antigens of the viral vector and without concomitant administration of the viral vector or concomitantly the synthetic nanocarriers that are attached to an immunosuppressant and that comprise no viral vector APC antigens of the viral vector and the viral vector; and (3) administering the first and second dosings to a subject according to an administration schedule that reduces an undesired humoral immune response to the viral vector and/or increases transgene or nucleic acid material expression or provides durable transgene or nucleic acid material expression, such as for at least one month or two months from the first dosing, wherein the dose of the viral vector of any one of the first and second dosings is at a dose lower than would otherwise be administered without the synthetic nanocarriers.

In an embodiment of any one of the methods provided herein, the method further comprises (4) a third dosing that comprises administering (d) the synthetic nanocarriers that are attached to an immunosuppressant and that comprise no viral vector APC antigens of the viral vector and without concomitant administration of the viral vector or concomitantly the synthetic nanocarriers that are attached to an immunosuppressant and that comprise no viral vector APC antigens of the viral vector and the viral vector; and (5) administering the third dosing to a subject also according to an administration schedule that reduces an undesired humoral immune response to the viral vector and/or increases transgene or nucleic acid material expression or provides durable transgene or nucleic acid material expression, such as for at least one month, two months or three months from the first dosing, wherein the dose of the viral vector of the third dosing is at a dose lower than would otherwise be administered without the synthetic nanocarriers.

In one embodiment of any one of the methods provided herein, the method further comprises (6) determining the administration schedule for the first and second dosings or first, second and third dosings that reduces an undesired humoral immune response to the viral vector and/ or increases transgene or nucleic acid material expression or provides durable transgene or nucleic acid material expression, such as for at least one month, two months or three months from the first dosing.

In one embodiment of any one of the methods provided herein, the lower dose of the viral vector of the first, second and/or third dosings is less than but at least ⅒ of the dose.

In one embodiment of any one of the methods provided herein, the dosings are or are about a month apart.

In an aspect, a method of manufacturing any one of the compositions or kits provided herein is provided. In one embodiment, the method of manufacturing comprises producing one or more doses or dosage forms of a viral vector and producing one or more doses or dosage forms of a population of synthetic nanocarriers that are attached to an immunosuppressant. In another embodiment of any one of the methods of manufacturing provided, the step of producing one or more doses or dosage forms of a population of synthetic nanocarriers that are attached to an immunosuppressant comprises attaching the immunosuppressant to synthetic nanocarriers. In another embodiment of any one of the methods of manufacturing provided, the method further comprises combining the one or more doses or dosage forms of the population of synthetic nanocarriers that are attached to an immunosuppressant and one or more doses or dosage forms of the viral vector in a kit.

In another aspect, a use of any one of the compositions or kits provided herein for the manufacture of a medicament for reducing an undesired immune response to a viral vector and/or increases transgene or nucleic acid material expression or provides durable transgene or nucleic acid material expression in a subject is provided. In one embodiment, the composition or kit comprises one or more doses or dosage forms comprising a population of synthetic nanocarriers that are attached to an immunosuppressant and one or more doses or dosage forms comprising a viral vector, wherein the population of synthetic nanocarriers that are attached to an immunosuppressant and viral vector are administered according to any one of the method provided herein. In some embodiments of any one of the uses provided herein, the population of synthetic nanocarriers that are attached to an immunosuppressant comprises no viral vector antigen-presenting cell (APC) presentable antigens of the viral vector. In some embodiments of any one of the uses provided herein, the composition or kit further comprises one or more doses or dosage forms comprising the population of synthetic nanocarriers that are attached to an immunosuppressant for use as one or more second or third dosings. In some embodiments of any one of the uses provided herein, the composition or kit further comprises one or more doses or dosage forms comprising the population of synthetic nanocarriers that are attached to an immunosuppressant as well as one or more doses or dosage forms comprising the viral vector at a lower dose, for use as one or more second or third dosings.

In another aspect, any one of the compositions or kits provided herein are provided for use in any one of the methods provided herein.

### BRIEF DESCRIPTION OF THE FIGURES

FIG. 1 shows the non-human primate study layout.

FIG. 2 shows anti-AAV8 IgG data through Day 84. Per the graph, the data points for days 0, 7, 14, 28, 56, and 84 move progressively in each group from left to right with d0 represented by the dot on the far left and d84 on the far right.

FIG. 3 shows Day 84 neutralizing antibody titer.

FIG. 4 shows Day 84 neutralizing antibody titer versus anti-AAV IgG.

FIG. 5 shows neutralizing antibody titers.

FIG. 6 shows transgene expression data through day 84.

FIG. 7 shows the study layout in BALB/c and C57BL/6 mice.

FIG. 8 demonstrates IgG dynamics. Per the graph, the data points for days 12, 19, 33, 47, 61, and 75 move progressively in each group from left to right with d12 represented by the dot on the far left and d75 on the far right.

FIG. 9 demonstrates IgG dynamics. Per the graph, the data points for days 12, 19, 33, 47, 61, and 75 move progressively in each group from left to right with d12 represented by the dot on the far left and d75 on the far right.

FIG. 10 demonstrates IgG dynamics. Per the graph, the data points for days 12, 19, 47, and 75 move progressively in each group from left to right with d12 represented by the dot on the far left and d75 on the far right.

FIG. 11 shows the study layout in BALB/c and C57BL/6 mice.

FIGS. 12A-12B show IgG dynamics. Per the graphs, the data points for days 12, 19, 33, 47, and 61 move progressively in each group from left to right with d12 represented by the dot on the far left and d61 on the far right.

FIG. 13 shows IgG dynamics. Per the graph, the data points for days 12, 19, 33, 47, and 61 move progressively in each group from left to right with d12 represented by the dot on the far left and d61 on the far right.

FIG. 14 shows the study layout in C57BL/6 mice.

FIG. 15 shows results with a high dose and monthly IMMTOR. Per the graph, the data points for days -1, 7, 12, 19, 27, 42, 55, 70, 84, 112, 140, and 168 move progressively in each group from left to right with d-1 represented by the dot on the far left and d168 on the far right.

FIG. 16 shows a study design.

FIG. 17 demonstrates increased and sustained SEAP expression in combination with IMMTOR. Normalized Single = sample SEAP Activity/Control group mean SEAP activity day 7.

FIGS. 18A-18E demonstrate that three monthly doses of IMMTOR mitigates anti-AAV IgG and IgM formation. Normalized signal = sample OD/negative control OD.

FIGS. 19A-19J demonstrate that three monthly doses of IMMTOR mitigates anti-AAV IgG and IgM formation.

FIG. 20 demonstrates that three monthly doses of IMMTOR are able to mitigate AAV8 neutralizing antibody development.

FIG. 21 shows that anti-AAV8 IgG OD correlate with anti-AAV8 neutralizing antibody titers.

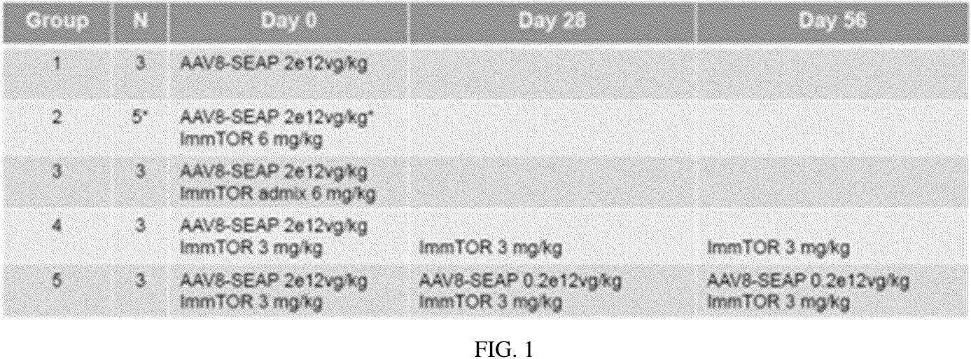

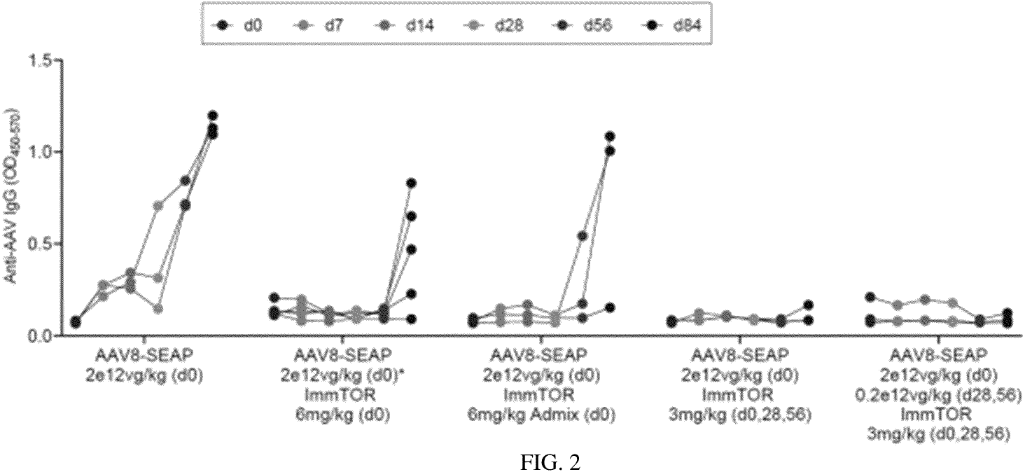

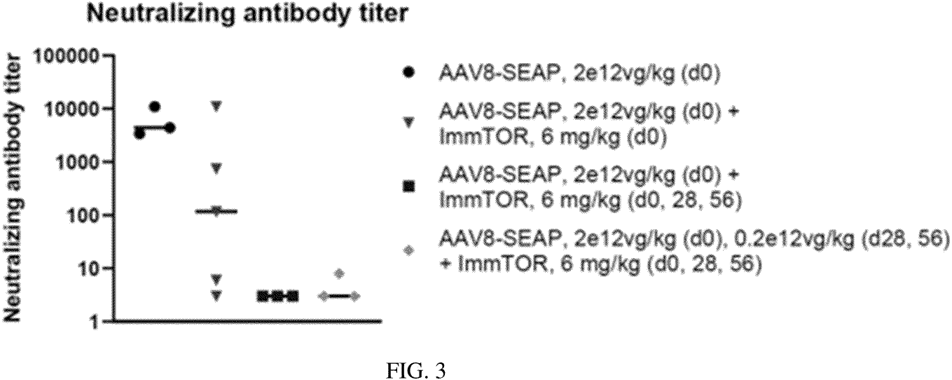

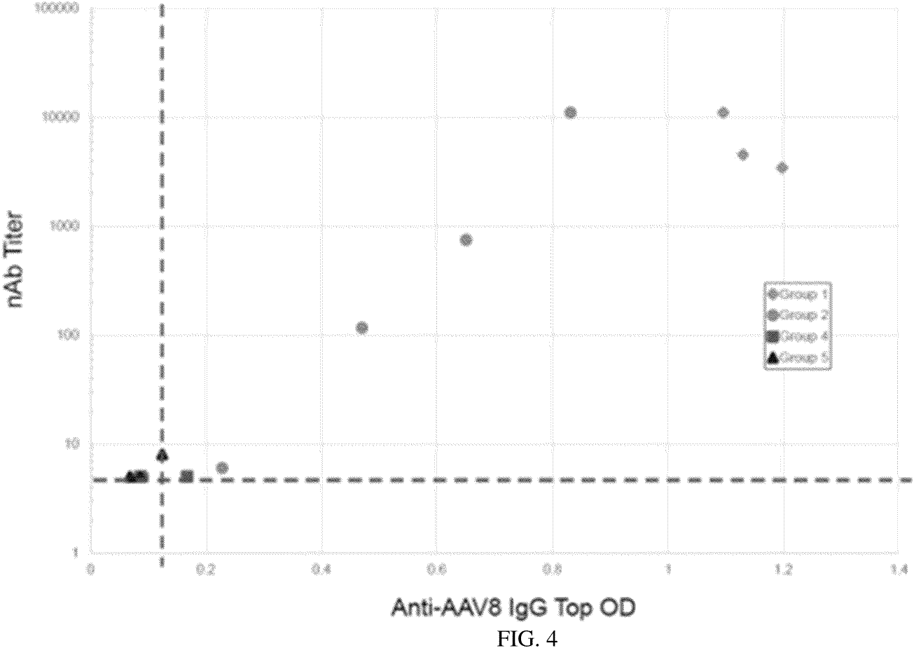

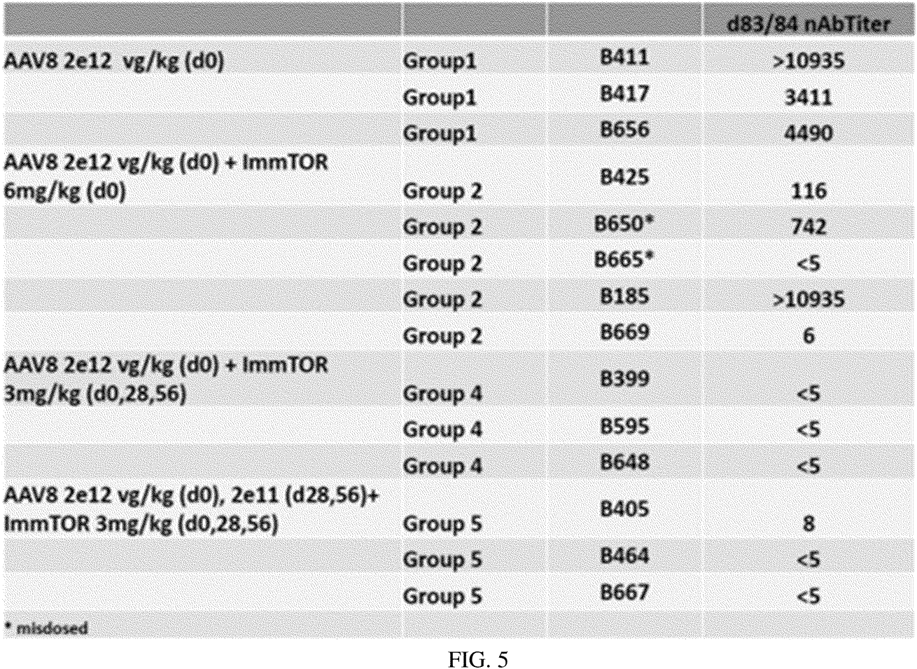

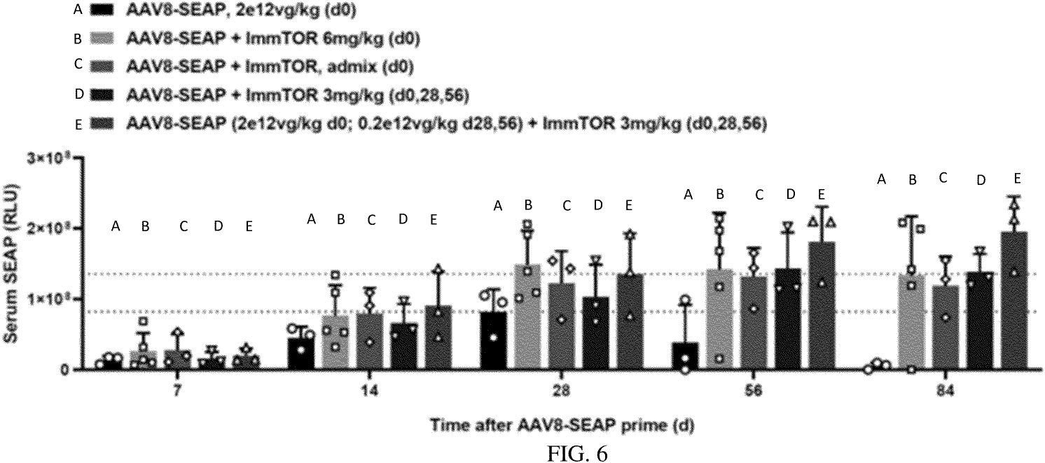

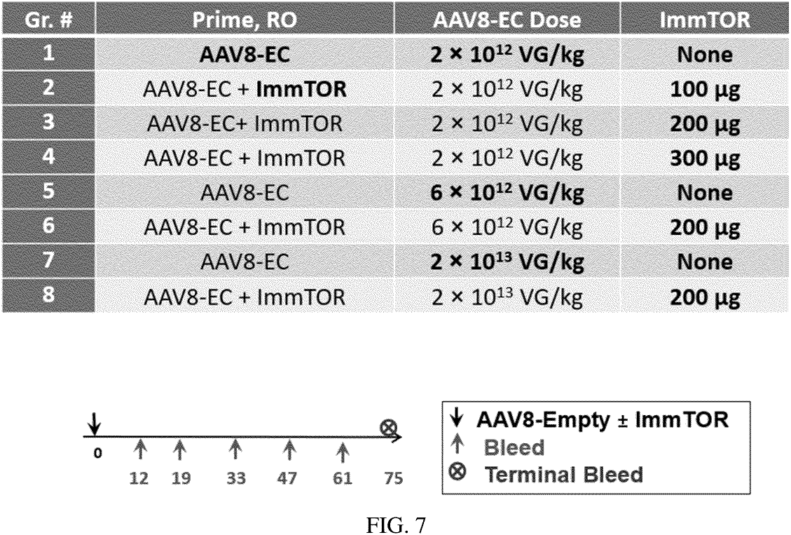

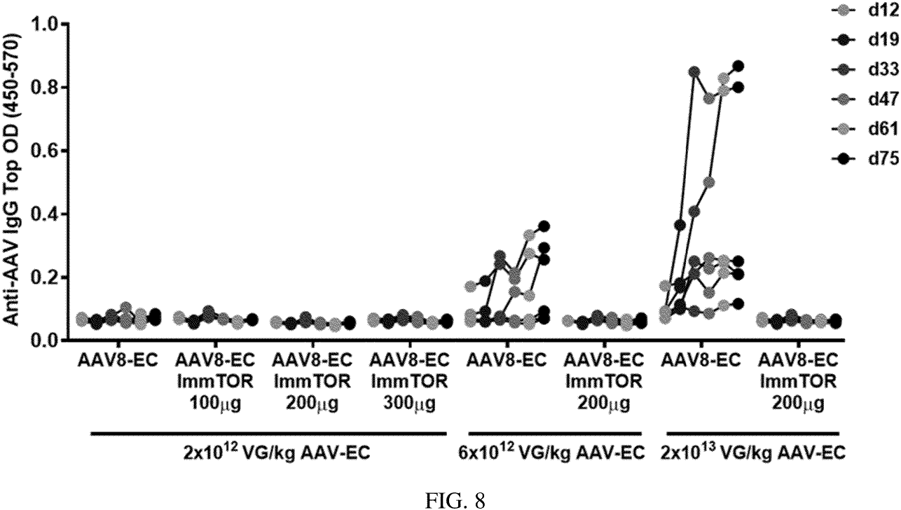

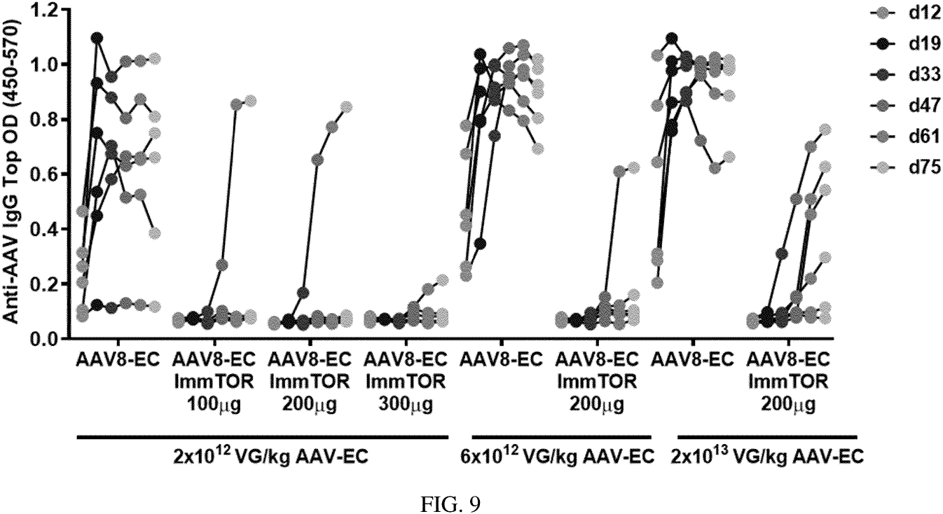

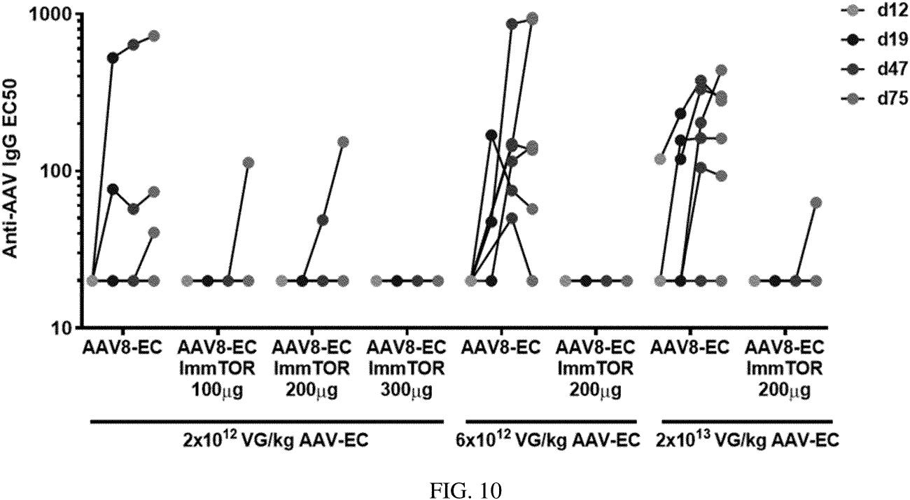

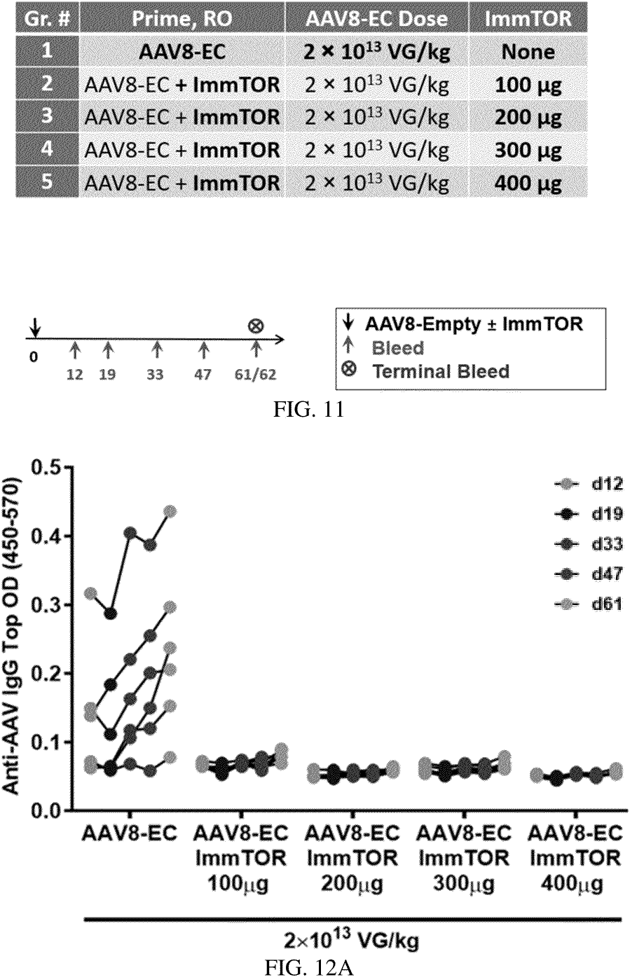

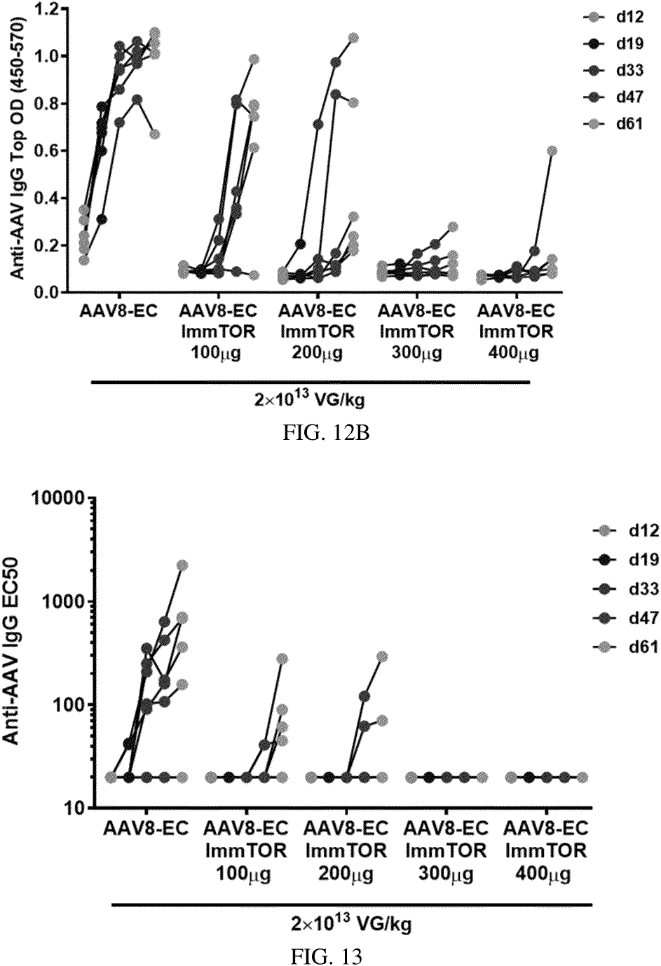

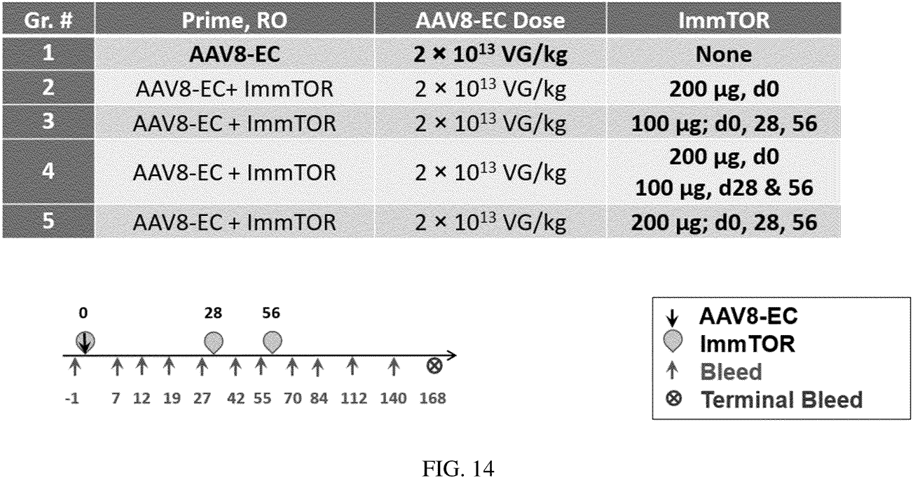

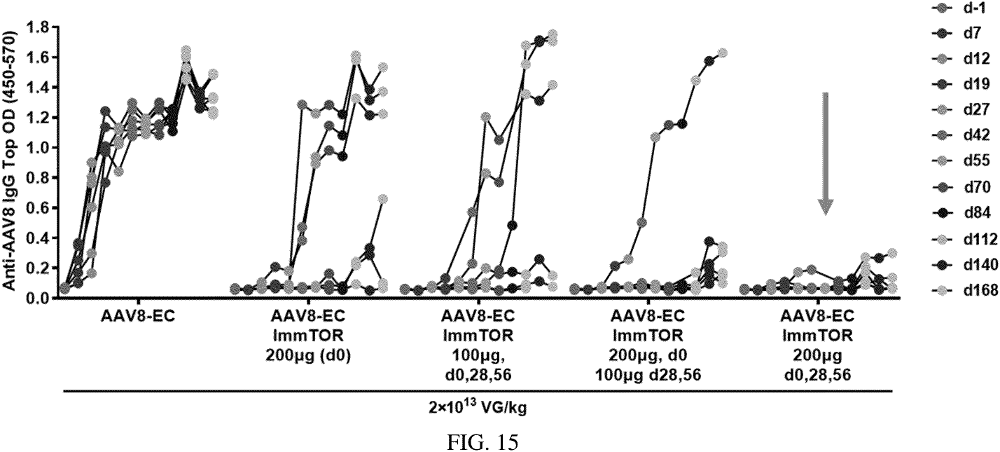

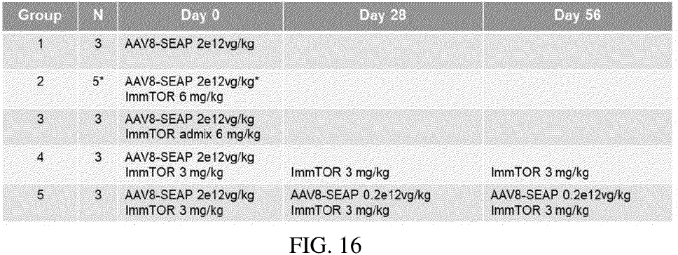

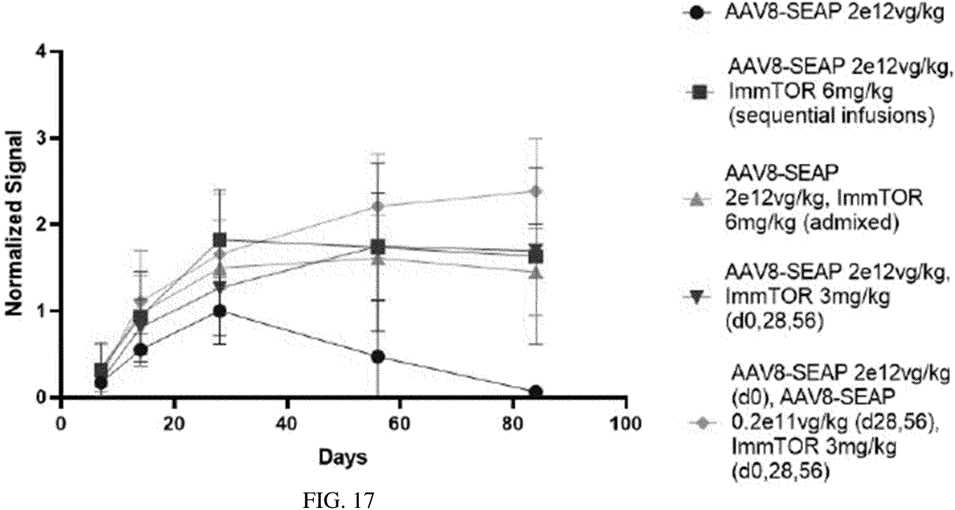

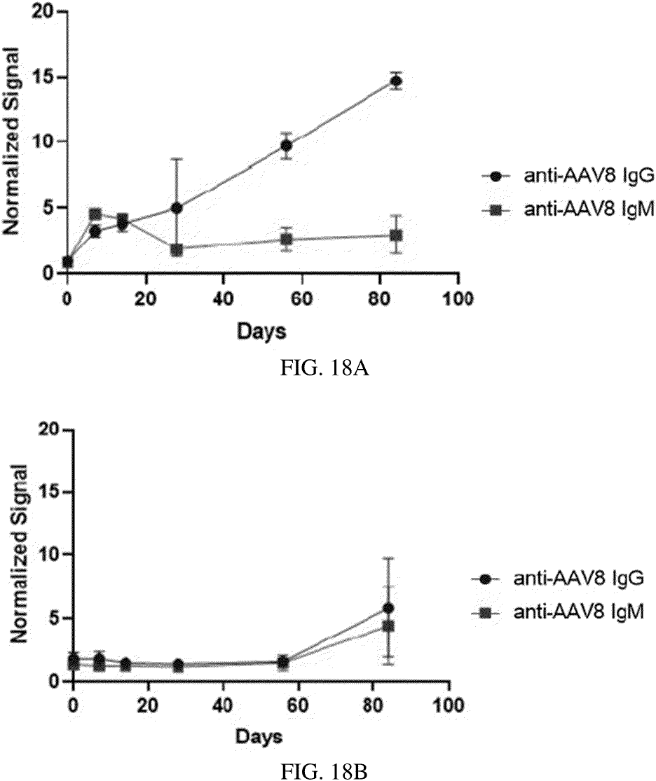

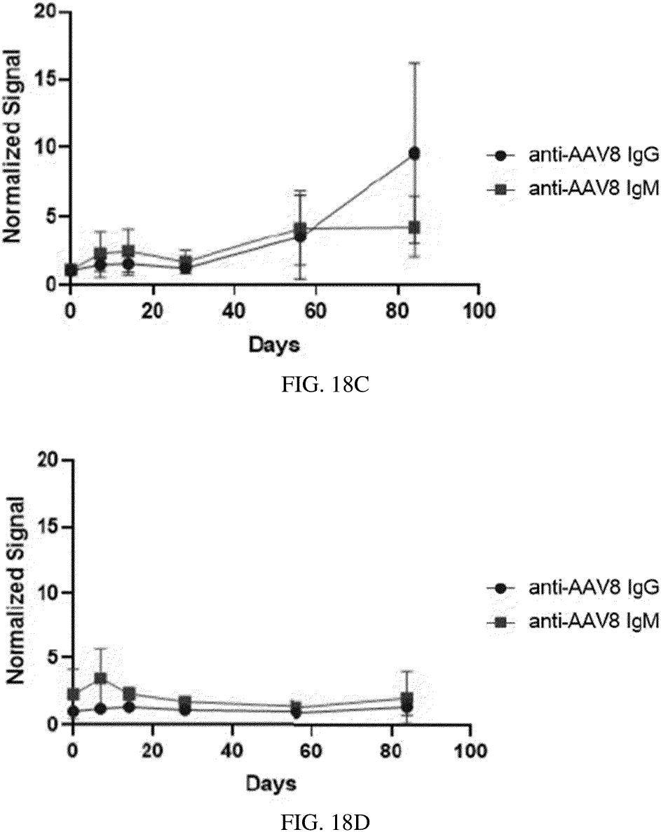

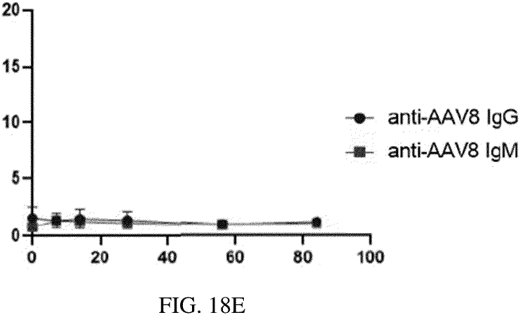

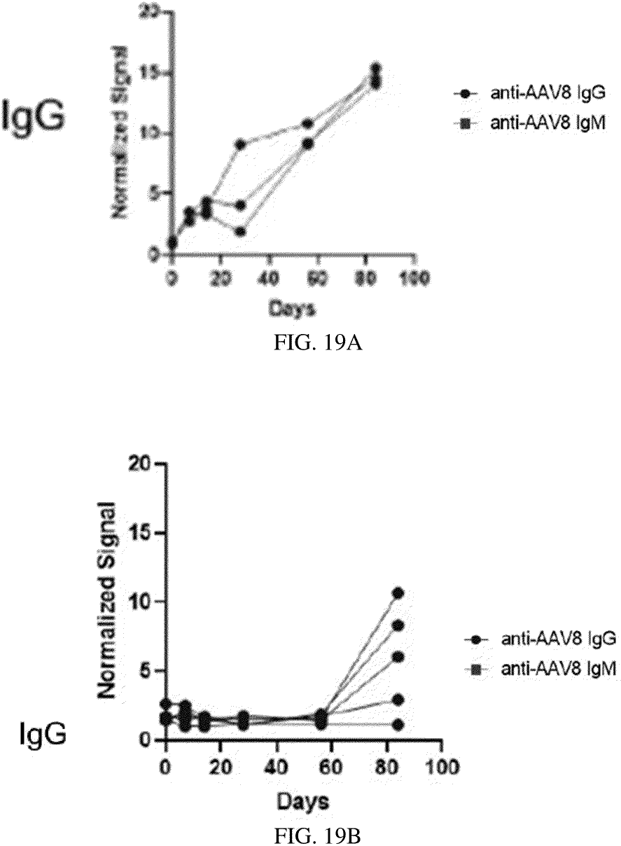

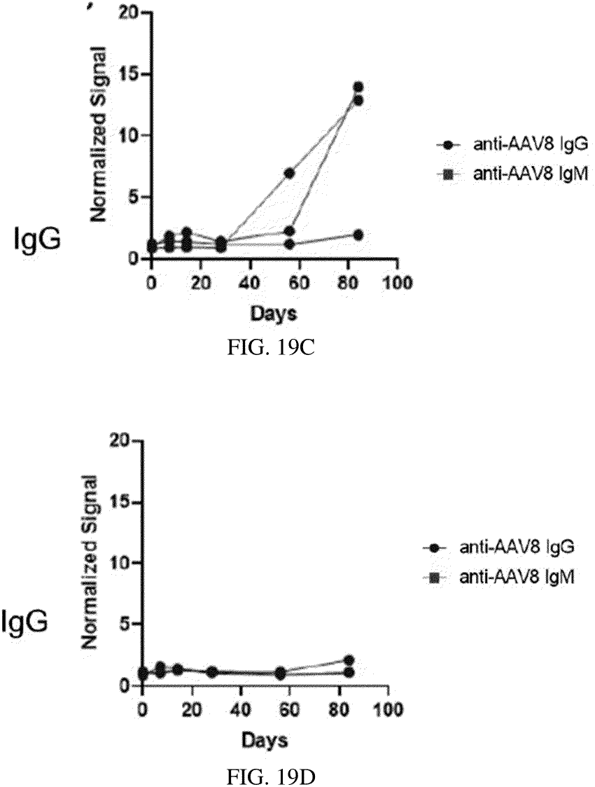

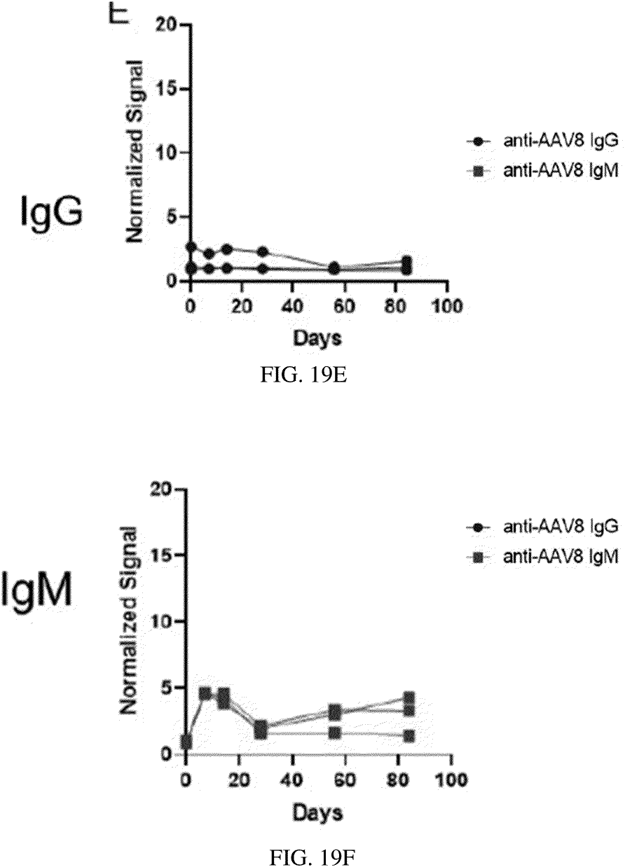

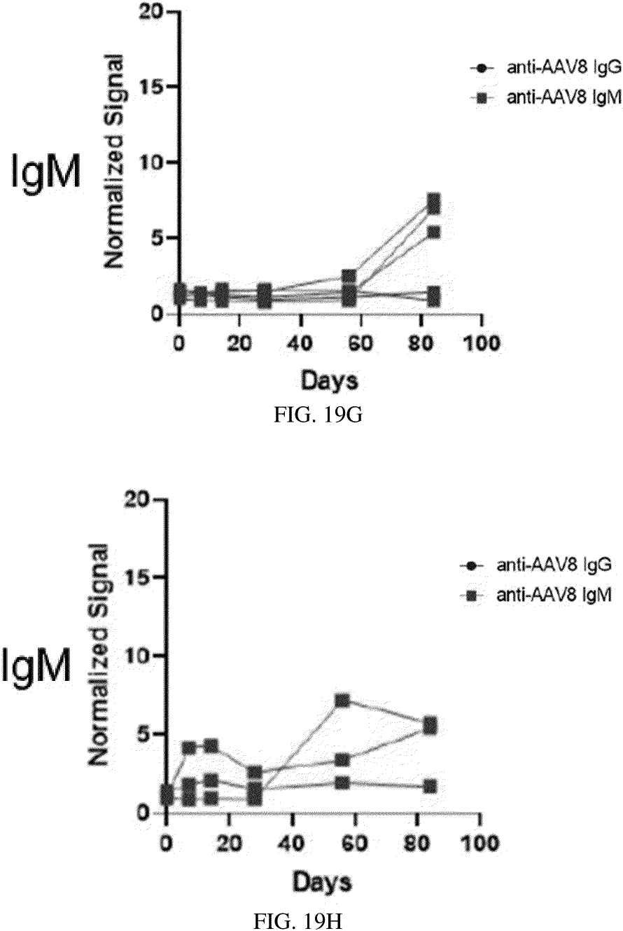

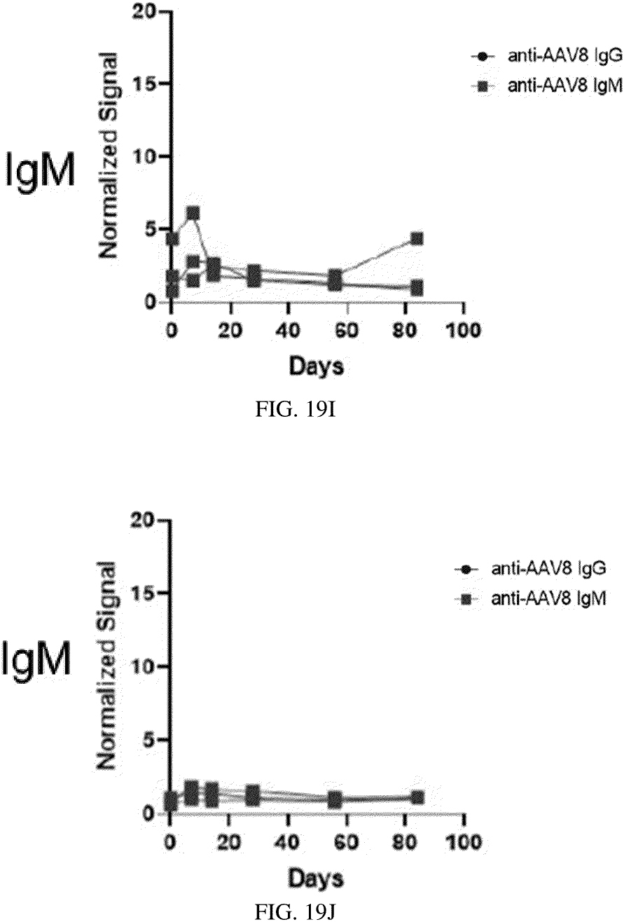

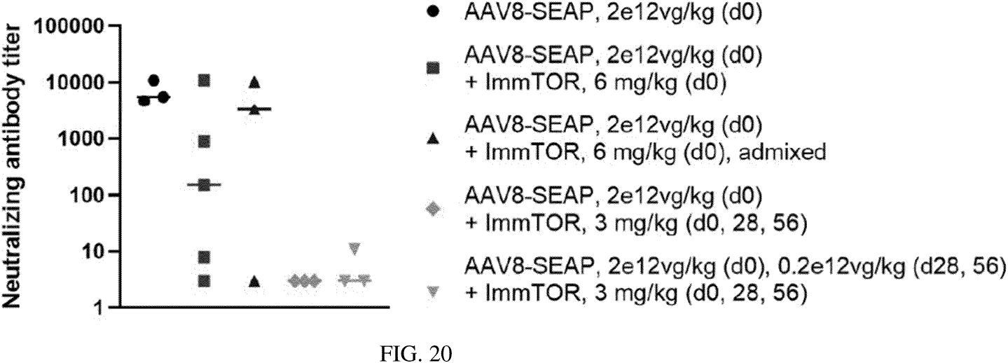

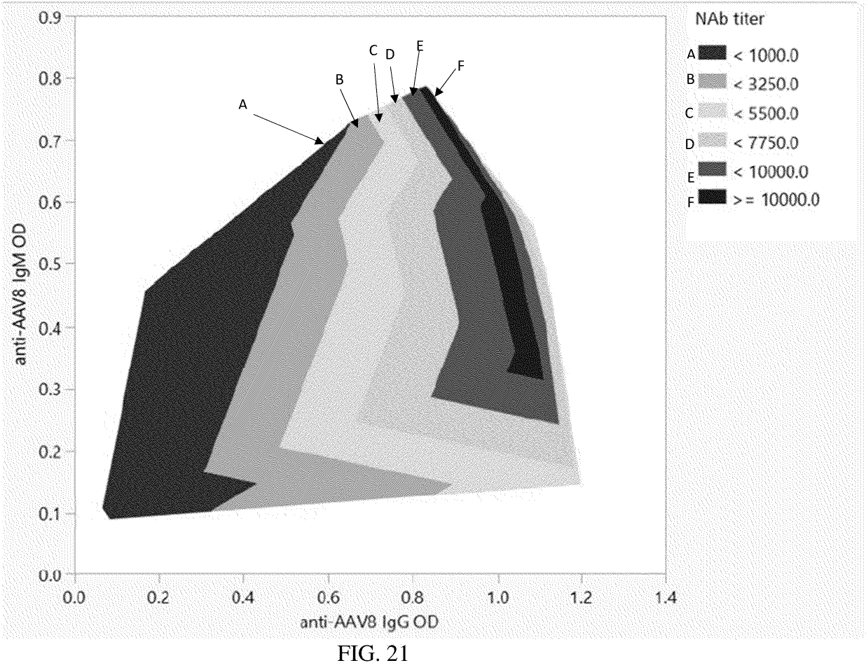

### DETAILED DESCRIPTION OF THE INVENTION

Before describing the present invention in detail, it is to be understood that this invention is not limited to particularly exemplified materials or process parameters as such may, of course, vary. It is also to be understood that the terminology used herein is for the purpose of describing particular embodiments of the invention only, and is not intended to be limiting of the use of alternative terminology to describe the present invention.

All publications, patents and patent applications cited herein, whether supra or infra, are hereby incorporated by reference in their entirety for all purposes.

As used in this specification and the appended claims, the singular forms “a,” “an” and “the” include plural referents unless the content clearly dictates otherwise. For example, reference to “a polymer” includes a mixture of two or more such molecules or a mixture of differing molecular weights of a single polymer species, reference to “a synthetic nanocarrier” includes a mixture of two or more such synthetic nanocarriers or a plurality of such synthetic nanocarriers, reference to “a RNA molecule” includes a mixture of two or more such RNA molecules or a plurality of such RNA molecules, reference to “an immunosuppressant” includes a mixture of two or more such materials or a plurality of immunosuppressant molecules, and the like.

As used herein, the term “comprise” or variations thereof such as “comprises” or “comprising” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein, the term “comprising” is inclusive and does not exclude additional, unrecited integers or method/process steps.

In embodiments of any one of the compositions and methods provided herein, “comprising” may be replaced with “consisting essentially of” or “consisting of”. The phrase “consisting essentially of” is used herein to require the specified integer(s) or steps as well as those which do not materially affect the character or function of the claimed invention. As used herein, the term “consisting” is used to indicate the presence of the recited integer (e.g. a feature, element, characteristic, property, method/process step or limitation) or group of integers (e.g. features, element, characteristics, properties, method/process steps or limitations) alone.

### A. Introduction

It has been surprisingly found that certain administration combinations can result in reduced anti-viral vector humoral immune response and/or increased or durable transgene or nucleic acid material expression. For example, the data provided herein demonstrate findings, including:
Higher levels of transgene expression with concomitant administration of viral vectors and synthetic nanocarriers attached to an immunosuppressant, indicating a first dose benefit of the synthetic nanocarriers on transgene expression.
Long-lasting and durable transgene expression with concomitant administration of viral vectors and synthetic nanocarriers attached to an immunosuppressant. In addition, it was found that lower doses of viral vector can be used and, in some embodiments, lead to increased transgene expression.
Administration of synthetic nanocarriers attached to an immunosuppressant can achieve robust and durable inhibition of anti-viral vector IgG antibodies. This effect was strengthened with repeat-dosing of the synthetic nanocarriers comprising the immunosuppressant.
Concomitant administration of synthetic nanocarriers attached to an immunosuppressant with viral vectors, such as monthly, can allow for repeated viral vector dosing at higher doses, such as at doses that may be clinically relevant for a human.

The invention will now be described in more detail below.

### B. Definitions

“Administering” or “administration” or “administer” means providing a material to a subject in a manner that is pharmacologically useful. The term is intended to include “causing to be administered” in some embodiments. “Causing to be administered” means causing, urging, encouraging, aiding, inducing or directing, directly or indirectly, another party to administer the material.

“Administration schedule” refers to administration of first dosings and second dosings and, optionally, third dosings according to a determined schedule. The schedule can include the number of dosings as well as the frequency of such dosings or interval between dosings. Such an administration schedule may include a number of parameters that are varied to achieve a particular objective, preferably reduction of an undesired humoral immune response to a viral vector antigen and/or increased or durable transgene or nucleic acid material expression. In embodiments, the administration schedule is any of the administration schedules as provided below in the Examples. In some embodiments, administration schedules according to the invention may be used to administer first and second dosings and, optionally, third dosings to one or more test subjects. Immune responses in these test subjects can then be assessed to determine whether or not the schedule was effective in reducing an undesired humoral immune response and/or increased or durable transgene or nucleic acid material expression. Whether or not a schedule had a desired effect can be determined using any of the methods provided herein or otherwise known in the art. For example, a sample may be obtained from a subject to which dosings provided herein have been administered according to a specific administration schedule in order to determine whether or not specific immune cells, cytokines, antibodies, etc. were reduced, generated, activated, etc. and/or specific proteins or expression products were increased, reduced or generated, etc. Useful methods for detecting the presence and/or number of immune cells include, but are not limited to, flow cytometric methods (e.g., FACS), ELISpot, proliferation responses, cytokine production, and immunohistochemistry methods. Useful methods for determining the level of protein, such as antibody, production are well known in the art and include the assays provided herein. Such assays include ELISA assays.

“Amount effective” in the context of a composition or dosage form for administration to a subject refers to an amount of the composition or dosage form that produces one or more desired immune responses or increased or durable transgene or nucleic acid material expression in the subject. Therefore, in some embodiments, an amount effective is any amount of a composition or dosage form provided herein that reduces an undesired humoral immune response and/or increases or provides durable transgene or nucleic acid material expression. This amount can be for in vitro or in vivo purposes. For in vivo purposes, the amount can be one that a clinician would believe may have a clinical benefit for a subject as provided herein.

Amounts effective can involve reducing the level of an undesired immune response, although in some embodiments, it involves preventing an undesired immune response altogether. Amounts effective can also involve delaying the occurrence of an undesired immune response. An amount that is effective can also be an amount that produces a desired therapeutic endpoint or a desired therapeutic result. Amounts effective, preferably, result in a reduction in an undesired humoral immune response in a subject specific to a viral vector and/or increases or provides durable transgene or nucleic acid material expression of a viral vector. Amounts effective, can also result in a tolerogenic immune response in a subject to an antigen, such as a viral vector antigen. In other embodiments, the amounts effective can involve enhancing the level of a desired response, such as a therapeutic endpoint or result. The achievement of any of the foregoing can be monitored by routine methods. An amount effective, such as one that has a therapeutic benefit, such as in a human, can be determined by a clinician or other medical practitioner.

In some embodiments of any one of the compositions and methods provided, the amount effective is one in which the desired immune response persists in the subject for at least 1 week, at least 2 weeks, at least 1 month, at least 2 months, at least 3 months, at least 4 months, at least 5 months, or longer. In other embodiments of any of the compositions and methods provided, the amount effective is one which produces a measurable desired response, for example, a measurable desired immune response, such as a decrease in a humoral immune response (e.g., to a specific antigen) and/or transgene or nucleic acid material expression response, for at least 1 week, at least 2 weeks, at least 1 month, at least 2 months, at least 3 months, at least 4 months, at least 5 months, or longer.

Amounts effective will depend, of course, on the particular subject being treated; the severity of a condition, disease or disorder; the individual patient parameters including age, physical condition, size and weight; the duration of the treatment; the nature of concurrent therapy (if any); the specific route of administration and like factors within the knowledge and expertise of the health practitioner. These factors are well known to those of ordinary skill in the art and can be addressed with no more than routine experimentation. It is generally preferred that a maximum dose be used, that is, the highest safe dose according to sound medical judgment. It will be understood by those of ordinary skill in the art, however, that a patient may insist upon a lower dose or tolerable dose for medical reasons, psychological reasons or for virtually any other reason.

Doses of the synthetic nanocarriers attached to an immunosuppressant and/or viral vector in the compositions of the invention can refer to the amount of the immunosuppressant attached to the synthetic nanocarriers and/or viral vector. Alternatively, the dose can be administered based on the number of synthetic nanocarriers that provide the desired amount of immunosuppressants.

“Anti-viral vector immune response” or “immune response against a viral vector” or the like refers to any undesired immune response against a viral vector. In some embodiments, the undesired immune response is an antigen-specific immune response against the viral vector or an antigen thereof. In some embodiments, the immune response is specific to a viral antigen of the viral vector. In other embodiments, the immune response is specific to an expression product, such as a protein or peptide, encoded by the transgene or nucleic acid material of the viral vector. In some embodiments, the immune response is specific to a viral antigen of the viral vector and not to a protein or peptide that is encoded by the transgene or nucleic acid material of the viral vector. The immune response may be an anti-viral vector antibody response, an anti-viral vector T cell immune response, such as a CD4+ T cell or CD8+ T cell immune response, or an anti-viral vector B cell immune response.

“Antigen” means a B cell antigen or T cell antigen. “Type(s) of antigens” means molecules that share the same, or substantially the same, antigenic characteristics. In some embodiments, antigens may be proteins, polypeptides, peptides, lipoproteins, glycolipids, polynucleotides, polysaccharides or are contained or expressed in cells. In some embodiments, such as when the antigens are not well defined or characterized, the antigens may be contained within a cell or tissue preparation, cell debris, cell exosomes, conditioned media, etc.

“Antigen-specific” refers to any immune response that results from the presence of the antigen, or portion thereof, or that generates molecules that specifically recognize or bind the antigen. In some embodiments, when the antigen is of a viral vector, antigen-specific may mean viral vector-specific. For example, where the immune response is antigen-specific antibody production, such as viral vector-specific antibody production, antibodies are produced that specifically bind the antigen (e.g., viral vector). As another example, where the immune response is antigen-specific B cell or CD4+ T cell proliferation and/or activity, the proliferation and/or activity results from recognition of the antigen, or portion thereof, alone or in complex with MHC molecules, B cells, etc.

“Assessing an immune response” refers to any measurement or determination of the level, presence or absence, reduction, increase in, etc. of an immune response in vitro or in vivo. Such measurements or determinations may be performed on one or more samples obtained from a subject. Such assessing can be performed with any of the methods provided herein or otherwise known in the art.

“Attach” or “Attached” or “Couple” or “Coupled” (and the like) means to chemically associate one entity (for example a moiety) with another. In some embodiments, the attaching is covalent, meaning that the attachment occurs in the context of the presence of a covalent bond between the two entities. In non-covalent embodiments, the non-covalent attaching is mediated by non-covalent interactions including but not limited to charge interactions, affinity interactions, metal coordination, physical adsorption, host-guest interactions, hydrophobic interactions, TT stacking interactions, hydrogen bonding interactions, van der Waals interactions, magnetic interactions, electrostatic interactions, dipole-dipole interactions, and/or combinations thereof. In embodiments, encapsulation is a form of attaching. In embodiments, the viral vector and synthetic nanocarriers attached to an immunosuppressant are not attached to one another, meaning that the viral vector and synthetic nanocarriers attached to an immunosuppressant are not subjected to a process specifically intended to chemically associate one with another.

An “at risk” subject is one in which a health practitioner believes has a chance of having a disease, disorder or condition or is one a health practitioner believes has a chance of experiencing an undesired humoral immune response as provided herein and would benefit from the compositions and methods provided. In some embodiments, the subjects are those that are expected to have an undesired humoral immune response to a viral vector.

“Average”, as used herein, refers to the arithmetic mean unless otherwise noted.

As used herein, the term “combination therapy” is intended to define therapies which comprise the use of a combination of two or more materials/agents (as defined above). Thus, references to “combination therapy”, “combinations” and the use of materials/agents “in combination” in this application may refer to materials/agents that are administered as part of the same overall treatment regimen. As such, the posology of each of the two or more materials/agents may differ: each may be administered at the same time or at different times. It will, therefore, be appreciated that the materials/agents of the combination may be administered sequentially (e.g., before or after) or simultaneously, either in the same pharmaceutical formulation (i.e., together), or in different pharmaceutical formulations (i.e., separately). Simultaneously in the same formulation is as a unitary formulation whereas simultaneously in different pharmaceutical formulations is non-unitary. The posologies of each of the two or more materials/agents in a combination therapy may also differ with respect to the route of administration.

“Concomitantly” means administering two or more materials/agents to a subject in a manner that is correlated in time, preferably sufficiently correlated in time so as to provide a modulation in an immune or physiologic response, and even more preferably the two or more materials/agents are administered in combination. In embodiments, concomitant administration may encompass administration of two or more materials/agents within a specified period of time, preferably within 1 month, more preferably within 1 week, still more preferably within 1 day, and even more preferably within 1 hour. In embodiments, the materials/agents may be repeatedly administered concomitantly, that is concomitant administration on more than one occasion, as may be provided in the Examples.

“Determining” or “determine” means to ascertain a factual relationship. Determining may be accomplished in a number of ways, including but not limited to performing experiments, or making projections. For instance, a dose of an immunosuppressant or viral vector may be determined by starting with a test dose and using known scaling techniques (such as allometric or isometric scaling) to determine the dose for administration. Such may also be used to determine a protocol or administration schedule as provided herein. In another embodiment, the dose may be determined by testing various doses in a subject, i.e. through direct experimentation based on experience and guiding data. In embodiments, “determining” or “determine” comprises “causing to be determined.” “Causing to be determined” means causing, urging, encouraging, aiding, inducing or directing or acting in coordination with an entity for the entity to ascertain a factual relationship; including directly or indirectly, or expressly or impliedly.

“Dose” refers to a specific quantity of a pharmacologically and/or immunologically active material for administration to a subject for a given time. In general, doses of the synthetic nanocarriers comprising an immunosuppressant and/or viral vectors in the methods and compositions of the invention refer to the amount of the synthetic nanocarriers comprising an immunosuppressant and/or viral vectors. Alternatively, the dose can be administered based on the number of synthetic nanocarriers that provide the desired amount of an immunosuppressant, in instances when referring to a dose of synthetic nanocarriers that comprise an immunosuppressant. When dose is used in the context of a repeated dosing, dose refers to the amount of each of the repeated doses, which may be the same or different.

“Dosing” means the administration of a pharmacologically and/or immunologically active material or combination of pharmacologically and/or immunologically active materials to a subject. The materials of a dosing may be administered concomitantly, such as simultaneously, in any one of the methods provided herein. The materials of a dosing may be administered admixed in the same composition in any one of the methods provided herein. The materials of a dosing may be administered separately in separate compositions in any one of the methods provided herein.

“Encapsulate” means to enclose at least a portion of a substance within a synthetic nanocarrier. In some embodiments, a substance is enclosed completely within a synthetic nanocarrier. In other embodiments, most or all of a substance that is encapsulated is not exposed to the local environment external to the synthetic nanocarrier. In other embodiments, no more than 50%, 40%, 30%, 20%, 10% or 5% (weight/weight) is exposed to the local environment. Encapsulation is distinct from absorption, which places most or all of a substance on a surface of a synthetic nanocarrier, and leaves the substance exposed to the local environment external to the synthetic nanocarrier. In any one of the methods or composition provided herein, the immunosuppressant may be encapsulated in the synthetic nanocarriers.

“Expression control sequences” are any sequences that can affect expression and can include promoters, enhancers, and operators. In one embodiment of any one of the methods or compositions provided, the expression control sequence is a promoter. In one embodiment of any one of the methods or compositions provided, the expression control sequence is a liver-specific promoter or a constitutive promoter. “Liver-specific promoters” are those that exclusively or preferentially result in expression in cells of the liver. “Constitutive promoters” are those that are thought of being generally active and not exclusive or preferential to certain cells. In any one of the nucleic acids or viral vectors provided herein the promoter may be any one of the promoters provided herein.

“Generating” means causing an action, such as an immune or physiologic response (e.g., a tolerogenic immune response) to occur, either directly oneself or indirectly.

“Identifying a subject” is any action or set of actions that allows a clinician to recognize a subject as one who may benefit from the methods, compositions or kits provided herein. Preferably, the identified subject is one who is in need of a therapeutic benefit from a viral vector and in which an undesired humoral immune response is expected to occur as provided herein. The action or set of actions may be either directly oneself or indirectly. In one embodiment of any one of the methods provided herein, the method further comprises identifying a subject in need of a method, composition or kit as provided herein.

“Immunosuppressant” means a compound that causes an APC to have an immunosuppressive effect (e.g., tolerogenic effect) or a T cell or a B cell to be suppressed. An immunosuppressive effect generally refers to the production or expression of cytokines or other factors by the APC that reduces, inhibits or prevents an undesired immune response or that promotes a desired immune response, such as a regulatory immune response. When the APC acquires an immunosuppressive function (under the immunosuppressive effect) on immune cells that recognize an antigen presented by this APC, the immunosuppressive effect is said to be specific to the presented antigen. Without being bound by any particular theory, it is thought that the immunosuppressive effect is a result of the immunosuppressant being delivered to the APC, preferably in the presence of an antigen. In one embodiment, the immunosuppressant is one that causes an APC to promote a regulatory phenotype in one or more immune effector cells. For example, the regulatory phenotype may be characterized by the inhibition of the production, induction, stimulation or recruitment of antigen-specific CD4+ T cells or B cells, the inhibition of the production of antigen-specific antibodies, the production, induction, stimulation or recruitment of Treg cells (e.g., CD4+CD25highFoxP3+ Treg cells), etc. This may be the result of the conversion of CD4+ T cells or B cells to a regulatory phenotype. This may also be the result of induction of FoxP3 in other immune cells, such as CD8+ T cells, macrophages and iNKT cells. In one embodiment, the immunosuppressant is one that affects the response of the APC after it processes an antigen. In another embodiment, the immunosuppressant is not one that interferes with the processing of the antigen. In a further embodiment, the immunosuppressant is not an apoptotic-signaling molecule. In another embodiment, the immunosuppressant is not a phospholipid.

Immunosuppressants include, but are not limited to, statins; mTOR inhibitors, such as rapamycin or a rapamycin analog; TGF-β signaling agents; TGF-β receptor agonists; histone deacetylase inhibitors, such as Trichostatin A; corticosteroids; inhibitors of mitochondrial function, such as rotenone; P38 inhibitors; NF-kβ inhibitors, such as 6Bio, Dexamethasone, TCPA-1, IKK VII; adenosine receptor agonists; prostaglandin E2 agonists (PGE2), such as Misoprostol; phosphodiesterase inhibitors, such as phosphodiesterase 4 inhibitor (PDE4), such as Rolipram; proteasome inhibitors; kinase inhibitors; G-protein coupled receptor agonists; G-protein coupled receptor antagonists; glucocorticoids; retinoids; cytokine inhibitors; cytokine receptor inhibitors; cytokine receptor activators; peroxisome proliferator-activated receptor antagonists; peroxisome proliferator-activated receptor agonists; histone deacetylase inhibitors; calcineurin inhibitors; phosphatase inhibitors; PI3KB inhibitors, such as TGX-221; autophagy inhibitors, such as 3-Methyladenine; aryl hydrocarbon receptor inhibitors; proteasome inhibitor I (PSI); and oxidized ATPs, such as P2X receptor blockers. Immunosuppressants also include IDO, vitamin D3, cyclosporins, such as cyclosporine A, aryl hydrocarbon receptor inhibitors, resveratrol, azathiopurine (Aza), 6-mercaptopurine (6-MP), 6-thioguanine (6-TG), FK506, sanglifehrin A, salmeterol, mycophenolate mofetil (MMF), aspirin and other COX inhibitors, niflumic acid, estriol, methotrexate and triptolide. In embodiments, the immunosuppressant may comprise any of the agents provided herein.

The immunosuppressant can be a compound that directly provides the immunosuppressive effect on APCs or it can be a compound that provides the immunosuppressive effect indirectly (i.e., after being processed in some way after administration). Immunosuppressants, therefore, include prodrug forms of any of the compounds provided herein.

In embodiments of any one of the methods, compositions or kits provided herein, the immunosuppressants provided herein are attached to synthetic nanocarriers. In preferable embodiments, the immunosuppressant is an element that is in addition to the material that makes up the structure of the synthetic nanocarrier. For example, in one embodiment, where the synthetic nanocarrier is made up of one or more polymers, the immunosuppressant is a compound that is in addition and attached to the one or more polymers. As another example, in one embodiment, where the synthetic nanocarrier is made up of one or more lipids, the immunosuppressant is again in addition and attached to the one or more lipids. In embodiments, such as where the material of the synthetic nanocarrier also results in an immunosuppressive effect, the immunosuppressant is an element present in addition to the material of the synthetic nanocarrier that results in an immunosuppressive effect.

Other exemplary immunosuppressants include, but are not limited, small molecule drugs, natural products, antibodies (e.g., antibodies against CD20, CD3, CD4), biologics-based drugs, carbohydrate-based drugs, nanoparticles, liposomes, RNAi, antisense nucleic acids, aptamers, methotrexate, NSAIDs; fingolimod; natalizumab; alemtuzumab; anti-CD3; tacrolimus (FK506); cytokines and growth factors, such as TGF-β and IL-10; etc. Further immunosuppressants, are known to those of skill in the art, and the invention is not limited in this respect.

“Load”, when attached to a synthetic nanocarrier, is the amount of the immunosuppressant attached to a synthetic nanocarrier based on the total dry recipe weight of materials in an entire synthetic nanocarrier (weight/weight). Generally, such a load is calculated as an average across a population of synthetic nanocarriers. In one embodiment, the load of the immunosuppressant on average across the synthetic nanocarriers is between 0.1% and 99%. In another embodiment, the load is between 0.1% and 50%. In yet another embodiment, the load of the immunosuppressant is between 0.1% and 20%. In a further embodiment, the load of the immunosuppressant is between 0.1% and 10%. In still a further embodiment, the load of the immunosuppressant is between 1% and 10%. In still a further embodiment, the load of the immunosuppressant is between 7% and 20%. In yet another embodiment, the load of the immunosuppressant is at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, at least 5%, at least 6%, at least at least 7%, at least 8%, at least 9%, at least 10%, at least 11%, at least 12%, at least 13%, at least 14%, at least 15%, at least 16%, at least 17%, at least 18%, at least 19% or at least 20%, at least 25%, or at least 30% on average across the population of synthetic nanocarriers. In yet a further embodiment, the load of the immunosuppressant is 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19% or 20% on average across the population of synthetic nanocarriers. In some embodiments of the above embodiments, the load of the immunosuppressant is no more than 25% or 30% on average across a population of synthetic nanocarriers. In embodiments, the load is calculated as may be described in the Examples or as otherwise known in the art.

“Maximum dimension of a synthetic nanocarrier” means the largest dimension of a nanocarrier measured along any axis of the synthetic nanocarrier. “Minimum dimension of a synthetic nanocarrier” means the smallest dimension of a synthetic nanocarrier measured along any axis of the synthetic nanocarrier. For example, for a spheroidal synthetic nanocarrier, the maximum and minimum dimension of a synthetic nanocarrier would be substantially identical, and would be the size of its diameter. Similarly, for a cuboidal synthetic nanocarrier, the minimum dimension of a synthetic nanocarrier would be the smallest of its height, width or length, while the maximum dimension of a synthetic nanocarrier would be the largest of its height, width or length. In an embodiment, a minimum dimension of at least 75%, preferably at least 80%, more preferably at least 90%, of the synthetic nanocarriers in a sample, based on the total number of synthetic nanocarriers in the sample, is equal to or greater than 100 nm. In an embodiment, a maximum dimension of at least 75%, preferably at least 80%, more preferably at least 90%, of the synthetic nanocarriers in a sample, based on the total number of synthetic nanocarriers in the sample, is equal to or less than 5 µm. Preferably, a minimum dimension of at least 75%, preferably at least 80%, more preferably at least 90%, of the synthetic nanocarriers in a sample, based on the total number of synthetic nanocarriers in the sample, is greater than 110 nm, more preferably greater than 120 nm, more preferably greater than 130 nm, and more preferably still greater than 150 nm. Aspects ratios of the maximum and minimum dimensions of synthetic nanocarriers may vary depending on the embodiment. For instance, aspect ratios of the maximum to minimum dimensions of the synthetic nanocarriers may vary from 1:1 to 1,000,000:1, preferably from 1:1 to 100,000:1, more preferably from 1:1 to 10,000:1, more preferably from 1:1 to 1000:1, still more preferably from 1:1 to 100:1, and yet more preferably from 1:1 to 10:1. Preferably, a maximum dimension of at least 75%, preferably at least 80%, more preferably at least 90%, of the synthetic nanocarriers in a sample, based on the total number of synthetic nanocarriers in the sample is equal to or less than 3 µm, more preferably equal to or less than 2 µm, more preferably equal to or less than 1 µm, more preferably equal to or less than 800 nm, more preferably equal to or less than 600 nm, and more preferably still equal to or less than 500 nm. In preferred embodiments, a minimum dimension of at least 75%, preferably at least 80%, more preferably at least 90%, of the synthetic nanocarriers in a sample, based on the total number of synthetic nanocarriers in the sample, is equal to or greater than 100 nm, more preferably equal to or greater than 120 nm, more preferably equal to or greater than 130 nm, more preferably equal to or greater than 140 nm, and more preferably still equal to or greater than 150 nm. Measurement of synthetic nanocarrier dimensions (e.g., effective diameter) may be obtained, in some embodiments, by suspending the synthetic nanocarriers in a liquid (usually aqueous) media and using dynamic light scattering (DLS) (e.g., using a Brookhaven ZetaPALS instrument). For example, a suspension of synthetic nanocarriers can be diluted from an aqueous buffer into purified water to achieve a final synthetic nanocarrier suspension concentration of approximately 0.01 to 0.1 mg/mL. The diluted suspension may be prepared directly inside, or transferred to, a suitable cuvette for DLS analysis. The cuvette may then be placed in the DLS, allowed to equilibrate to the controlled temperature, and then scanned for sufficient time to acquire a stable and reproducible distribution based on appropriate inputs for viscosity of the medium and refractive indicies of the sample. The effective diameter, or mean of the distribution, is then reported. Determining the effective sizes of high aspect ratio, or non-spheroidal, synthetic nanocarriers may require augmentative techniques, such as electron microscopy, to obtain more accurate measurements. “Dimension” or “size” or “diameter” of synthetic nanocarriers means the mean of a particle size distribution, for example, obtained using dynamic light scattering.

“Non-methoxy-terminated polymer” means a polymer that has at least one terminus that ends with a moiety other than methoxy. In some embodiments, the polymer has at least two termini that ends with a moiety other than methoxy. In other embodiments, the polymer has no termini that ends with methoxy. “Non-methoxy-terminated, pluronic polymer” means a polymer other than a linear pluronic polymer with methoxy at both termini. Polymeric nanoparticles as provided herein can comprise non-methoxy-terminated polymers or non-methoxy-terminated, pluronic polymers.

“Pharmaceutically acceptable excipient” or “pharmaceutically acceptable carrier” means a pharmacologically inactive material used together with a pharmacologically active material to formulate the compositions. Pharmaceutically acceptable excipients comprise a variety of materials known in the art, including but not limited to saccharides (such as glucose, lactose, and the like), preservatives such as antimicrobial agents, reconstitution aids, colorants, saline (such as phosphate buffered saline), and buffers.

“Providing” means an action or set of actions that an individual performs that supply a needed item or set of items or methods for practicing of the present invention. The action or set of actions may be taken either directly oneself or indirectly.

“Providing a subject” is any action or set of actions that causes a clinician to come in contact with a subject and administer a composition provided herein thereto or to perform a method provided herein thereupon. In some embodiments, the subject is one who is in need of viral vector administration and antigen-specific immune tolerance thereto or any one of the desired results as provided herein. The action or set of actions may be taken either directly oneself or indirectly. In one embodiment of any one of the methods provided herein, the method further comprises providing a subject.

“Subject” means animals, including warm blooded mammals such as humans and primates; avians; domestic household or farm animals such as cats, dogs, sheep, goats, cattle, horses and pigs; laboratory animals such as mice, rats and guinea pigs; fish; reptiles; zoo and wild animals; and the like. As used herein, a subject may be in one need of any one of the methods or compositions provided herein. In some embodiments, the subject has or is suspected of having organic acidemia. In some embodiments, the subject is at risk of developing organic acidemia. In some embodiments, the organic acidemia is methylmalonic acidemia. In some embodiments, the organic academia is juvenile methylmalonic acidemia. In some embodiments, the subject is a pediatric or juvenile subject, e.g., is less than 18, less than 16, less than 15, less than 14, less than 13, less than 12, less than 11, less than 10, less than 9, less than 8, less than 7, less than 6, less than 5, less than 4, less than 3 year old, or less than 2 year old. In some embodiments, the subject is an adult subject.

“Synthetic nanocarrier(s)” means a discrete object that is not found in nature, and that possesses at least one dimension that is less than or equal to 5 microns in size. Albumin nanoparticles are generally included as synthetic nanocarriers, however in certain embodiments the synthetic nanocarriers do not comprise albumin nanoparticles. In embodiments, synthetic nanocarriers do not comprise chitosan. In other embodiments, synthetic nanocarriers are not lipid-based nanoparticles. In further embodiments, synthetic nanocarriers do not comprise a phospholipid.

A synthetic nanocarrier can be, but is not limited to, one or a plurality of lipid-based nanoparticles (also referred to herein as lipid nanoparticles, i.e., nanoparticles where the majority of the material that makes up their structure are lipids), polymeric nanoparticles, metallic nanoparticles, surfactant-based emulsions, dendrimers, buckyballs, nanowires, virus-like particles (i.e., particles that are primarily made up of viral structural proteins but that are not infectious or have low infectivity), peptide or protein-based particles (also referred to herein as protein particles, i.e., particles where the majority of the material that makes up their structure are peptides or proteins) (such as albumin nanoparticles) and/or nanoparticles that are developed using a combination of nanomaterials such as lipid-polymer nanoparticles. Synthetic nanocarriers may be a variety of different shapes, including but not limited to spheroidal, cuboidal, pyramidal, oblong, cylindrical, toroidal, and the like. Synthetic nanocarriers according to the invention comprise one or more surfaces. Exemplary synthetic nanocarriers that can be adapted for use in the practice of the present invention comprise: (1) the biodegradable nanoparticles disclosed in U.S. Pat. 5,543,158 to Gref et al., (2) the polymeric nanoparticles of Published U.S. Pat. Application 20060002852 to Saltzman et al., (3) the lithographically constructed nanoparticles of Published U.S. Pat. Application 20090028910 to DeSimone et al., (4) the disclosure of WO 2009/051837 to von Andrian et al., (5) the nanoparticles disclosed in Published U.S. Pat. Application 2008/0145441 to Penades et al., (6) the protein nanoparticles disclosed in Published U.S. Pat. Application 20090226525 to de los Rios et al., (7) the virus-like particles disclosed in published U.S. Pat. Application 20060222652 to Sebbel et al., (8) the nucleic acid attached virus-like particles disclosed in published U.S. Pat. Application 20060251677 to Bachmann et al., (9) the virus-like particles disclosed in WO2010047839A1 or WO2009106999A2, (10) the nanoprecipitated nanoparticles disclosed in P. Paolicelli et al., “Surface-modified PLGA-based Nanoparticles that can Efficiently Associate and Deliver Virus-like Particles″ Nanomedicine. 5(6):843-853 (2010), (11) apoptotic cells, apoptotic bodies or the synthetic or semisynthetic mimics disclosed in U.S. Publication 2002/0086049, or (12) those of Look et al., Nanogel-based delivery of mycophenolic acid ameliorates systemic lupus erythematosus in mice” J. Clinical Investigation 123(4):1741-1749(2013). In embodiments, synthetic nanocarriers may possess an aspect ratio greater than or equal to 1:1, 1:1.2, 1:1.5, 1:2, 1:3, 1:5, 1:7, or greater than 1:10.

Synthetic nanocarriers according to the invention that have a minimum dimension of equal to or less than about 100 nm, preferably equal to or less than 100 nm, do not comprise a surface with hydroxyl groups that activate complement or alternatively comprise a surface that consists essentially of moieties that are not hydroxyl groups that activate complement. In a preferred embodiment, synthetic nanocarriers according to the invention that have a minimum dimension of equal to or less than about 100 nm, preferably equal to or less than 100 nm, do not comprise a surface that substantially activates complement or alternatively comprise a surface that consists essentially of moieties that do not substantially activate complement. In a more preferred embodiment, synthetic nanocarriers according to the invention that have a minimum dimension of equal to or less than about 100 nm, preferably equal to or less than 100 nm, do not comprise a surface that activates complement or alternatively comprise a surface that consists essentially of moieties that do not activate complement. In embodiments, synthetic nanocarriers exclude virus-like particles. In embodiments, synthetic nanocarriers may possess an aspect ratio greater than or equal to 1:1, 1:1.2, 1:1.5, 1:2, 1:3, 1:5, 1:7, or greater than 1:10.

“Transgene or nucleic acid material expression” refers to the level of the transgene or nucleic acid material expression product of a viral vector in a subject, the transgene or nucleci acid material being delivered by the viral vector. In some embodiments, the level of expression may be determined by measuring transgene protein concentrations in various tissues or systems of interest in the subject. Alternatively, when the expression product is a nucleic acid, the level of expression may be measured by nucleic acid products. Increasing expression can be determined, for example, by measuring the amount of the expression product in a sample obtained from a subject and comparing it to a prior sample. Durability of expression may be measured by similar or other methods that would be apparent to one of ordinary skill in the art. The sample may be a tissue sample. In some embodiments, the expression product can be measured using flow cytometry.

“Undesired humoral immune response” refers to any undesired humoral immune response that results from exposure to an antigen, promotes or exacerbates a disease, disorder or condition provided herein (or a symptom thereof), or is symptomatic of a disease, disorder or condition provided herein. Such immune responses generally have a negative impact on a subject’s health or is symptomatic of a negative impact on a subject’s health. Undesired humoral immune responses include antigen-specific antibody production, antigen-specific B cell proliferation and/or activity or antigen-specific CD4+ T cell proliferation and/or activity. Generally, herein, these undesired immune responses are specific to a viral vector and counteract the beneficial effects desired of administration with the viral vector.

“Viral vector” means a vector construct with viral components, such as capsid and/or coat proteins, that has been adapted to comprise and deliver a transgene or nucleic acid material that encodes a therapeutic, such as a therapeutic protein, which transgene or nucleic acid material can be expressed as provided herein. “Expressed” or “expression” or the like refers to the synthesis of a functional (i.e., physiologically active for the desired purpose) product after the transgene or nucleic acid material is transduced into a cell and processed by the transduced cell. Such a product is also referred to herein as an “expression product”. Viral vectors can be based on, without limitation, adeno-associated viruses, such as AAV8 or AAV2. Thus, an AAV vector provided herein is a viral vector based on an AAV, such as AAV8 or AAV2, and has viral components, such as a capsid and/or coat protein, therefrom that can package for delivery the transgene or nucleic acid material. In some embodiments, the viral vector is a “chimeric viral vector”. In such embodiments, this means that the viral vector is made up of viral components that are derived from more than one virus or viral vector.

“Viral vector APC presentable antigen” means an antigen that is associated with a viral vector (i.e., the viral vector or a fragment thereof that can generate an immune response against the viral vector (e.g., the production of anti-viral vector-specific antibodies)). Generally, viral vector antigen-presenting cell (APC) presentable antigens can be presented for recognition by the immune system (e.g., cells of the immune system, such as presented by antigen presenting cells, including but not limited to dendritic cells, B cells or macrophages). The viral vector APC presentable antigen can be presented for recognition by, for example, T cells. Such antigens may be recognized by and trigger an immune response in a T cell via presentation of an epitope of the antigen bound to a Class I or Class II major histocompatability complex molecule (MHC). Viral vector APC presentable antigens generally include proteins, polypeptides, peptides, polynucleotides, etc., or are contained or expressed in, on or by cells. The viral vector antigens, in some embodiments, comprise MHC Class I-restricted epitopes and/or MHC Class II-restricted epitopes and/or B cell epitopes. In some embodiments, one or more tolerogenic immune responses specific to the viral vector result with the methods, compositions or kits provided herein. In embodiments, populations of the synthetic nanocarriers comprise no added viral vector APC presentable antigens, meaning that no substantial amounts of viral vector APC presentable antigens are intentionally added to the synthetic nanocarriers during the manufacturing thereof.

### C. Compositions Useful in the Practice of the Methods

The methods and related compositions provided herein, therefore, can be used for subjects in need of treatment with a viral vector, such as Methylmalonic Acidemia (MMA) or Ornithine Transcarbamylase (OTC) Deficiency. Any one of the methods or compositions provided herein can be for the treatment of MMA or OTC Deficiency.

MMA is a rare monogenic disorder in which the body cannot break down certain proteins and fats. This metabolic disease may lead to hyperammonemia and is associated with long-term complications including feeding problems, intellectual disability, chronic kidney disease and inflammation of the pancreas. Symptoms of MMA usually appear in early infancy and vary from mild to life-threatening. Without treatment, this disorder can lead to coma and in some cases death.

OTC deficiency is an X-linked genetic disorder caused by genetic mutations in the OTC gene, which is critical for proper function of the urea cycle. Individuals with OTC experience accumulation of excessive levels of ammonia in the blood. The most severe form of the disorder presents within the first few days of life and is characterized by an inability to control body temperature and breathing rate, seizures, coma, developmental delays and intellectual disability. Because the disorder is X-linked, males are most often affected by the severe form of the disease. Less severe forms of the disorder are characterized by delirium, erratic behavior, aversion to high protein foods, vomiting and seizures. Most approved therapies are focused on reducing the amount of ammonia in the blood and are not curative. Currently, the only curative approach is liver transplantation at an early age, which can be associated with severe side effects and complications. The dosings provided herein can be used in the treatment of any one of the disease or disorders provided herein.

The transgene or nucleic acid material, such as of the viral vectors, provided herein may encode any protein or portion thereof beneficial to a subject, such as one with a disease or disorder. In embodiments, the subject has or is suspected of having a disease or disorder whereby the subject’s endogenous version of the protein is defective or produced in limited amounts or not at all. The subject may be one with any one of the diseases or disorders as provided herein, and the transgene or nucleic acid material is one that encodes any one of the therapeutic proteins or portion thereof as provided herein. The transgene or nucleic acid material provided herein may encode a functional version of any protein that through some defect in the endogenous version of which in a subject (including a defect in the expression of the endogenous version) results in a disease or disorder in the subject. Examples of such diseases or disorders include, but are not limited to, urea cycle enzyme defects, such as ornithine transcarbamylase synthetase deficiency (OTCd). It follows that therapeutic proteins encoded by the transgene or nucleic acid material include ornithine transcarbamylase synthetase (OTC). Other examples of such diseases or disorders include, but are not limited to, organic acidemias, such as methylmalonic acidemia (MMA). It follows that therapeutic proteins encoded by the transgene or nucleic acid material also include methylmalonyl-CoA mutase (MUT), including any wild-type version of MUT, an enzyme that is frequently mutated in cases of MMA.

The sequence of a transgene or nucleic acid material may also include an expression control sequence. Expression control sequences include promoters, enhancers, and operators, and are generally selected based on the expression systems in which the expression construct is to be utilized. In some embodiments, promoter and enhancer sequences are selected for the ability to increase gene expression, while operator sequences may be selected for the ability to regulate gene expression. The transgene may also include sequences that facilitate, and preferably promote, homologous recombination in a host cell. The transgene may also include sequences that are necessary for replication in a host cell.

Exemplary expression control sequences include liver-specific promoter sequences and constitutive promoter sequences, such as any one that may be provided herein. Generally, promoters are operatively linked upstream (i.e., 5′) of the sequence coding for a desired expression product. The transgene also may include a suitable polyadenylation sequence operably linked downstream (i.e., 3′) of the coding sequence.

Viruses have evolved specialized mechanisms to transport their genomes inside the cells that they infect; viral vectors based on such viruses can be tailored to transduce cells to specific applications. Examples of viral vectors that may be used as provided herein are known in the art or described herein. Suitable viral vectors include, for instance, adeno-associated virus (AAV)-based vectors.

The viral vectors provided herein can be based on adeno-associated viruses (AAVs). AAV vectors have been of particular interest for use in therapeutic applications such as those described herein. AAV is a DNA virus, which is not known to cause human disease. Generally, AAV requires co-infection with a helper virus (e.g., an adenovirus or a herpes virus), or expression of helper genes, for efficient replication. For a description of AAV-based vectors, see, for example, U.S. Pat. Nos. 8,679,837, 8,637,255, 8,409,842, 7,803,622, and 7,790,449, and U.S. Publication Nos. 20150065562, 20140155469, 20140037585, 20130096182, 20120100606, and 20070036757. The AAV vectors may be recombinant AAV vectors. The AAV vectors may also be self-complementary (sc) AAV vectors, which are described, for example, in U.S. Pat. Publications 2007/01110724 and 2004/0029106, and U.S. Pat. Nos. 7,465,583 and 7,186,699.

The adeno-associated virus on which a viral vector is based may be of a specific serotype, such as AAV8 or AAV2. In some embodiments of any one of the methods or compositions provided herein, therefore, the AAV vector is an AAV8 or AAV2 vector.

A wide variety of synthetic nanocarriers can be used to attach to immunosuppressants of the dosings. In some embodiments, synthetic nanocarriers are spheres or spheroids. In some embodiments, synthetic nanocarriers are flat or plate-shaped. In some embodiments, synthetic nanocarriers are cubes or cubic. In some embodiments, synthetic nanocarriers are ovals or ellipses. In some embodiments, synthetic nanocarriers are cylinders, cones, or pyramids.

In some embodiments, it is desirable to use a population of synthetic nanocarriers that is relatively uniform in terms of size or shape so that each synthetic nanocarrier has similar properties. For example, at least 80%, at least 90%, or at least 95% of the synthetic nanocarriers, based on the total number of synthetic nanocarriers, may have a minimum dimension or maximum dimension that falls within 5%, 10%, or 20% of the average diameter or average dimension of the synthetic nanocarriers.

Synthetic nanocarriers can be solid or hollow and can comprise one or more layers. In some embodiments, each layer has a unique composition and unique properties relative to the other layer(s). To give but one example, synthetic nanocarriers may have a core/shell structure, wherein the core is one layer (e.g., a polymeric core) and the shell is a second layer (e.g., a lipid bilayer or monolayer). Synthetic nanocarriers may comprise a plurality of different layers.

In some embodiments, synthetic nanocarriers may optionally comprise one or more lipids. In some embodiments, a synthetic nanocarrier may comprise a liposome. In some embodiments, a synthetic nanocarrier may comprise a lipid bilayer. In some embodiments, a synthetic nanocarrier may comprise a lipid monolayer. In some embodiments, a synthetic nanocarrier may comprise a micelle. In some embodiments, a synthetic nanocarrier may comprise a core comprising a polymeric matrix surrounded by a lipid layer (e.g., lipid bilayer, lipid monolayer, etc.). In some embodiments, a synthetic nanocarrier may comprise a non-polymeric core (e.g., metal particle, quantum dot, ceramic particle, bone particle, viral particle, proteins, nucleic acids, carbohydrates, etc.) surrounded by a lipid layer (e.g., lipid bilayer, lipid monolayer, etc.).

In other embodiments, synthetic nanocarriers may comprise metal particles, quantum dots, ceramic particles, etc. In some embodiments, a non-polymeric synthetic nanocarrier is an aggregate of non-polymeric components, such as an aggregate of metal atoms (e.g., gold atoms).

In some embodiments, synthetic nanocarriers may optionally comprise one or more amphiphilic entities. In some embodiments, an amphiphilic entity can promote the production of synthetic nanocarriers with increased stability, improved uniformity, or increased viscosity. In some embodiments, amphiphilic entities can be associated with the interior surface of a lipid membrane (e.g., lipid bilayer, lipid monolayer, etc.). Many amphiphilic entities known in the art are suitable for use in making synthetic nanocarriers in accordance with the present invention. Such amphiphilic entities include, but are not limited to, phosphoglycerides; phosphatidylcholines; dipalmitoyl phosphatidylcholine (DPPC); dioleylphosphatidyl ethanolamine (DOPE); dioleyloxypropyltriethylammonium (DOTMA); dioleoylphosphatidylcholine; cholesterol; cholesterol ester; diacylglycerol; diacylglycerolsuccinate; diphosphatidyl glycerol (DPPG); hexanedecanol; fatty alcohols such as polyethylene glycol (PEG); polyoxyethylene-9-lauryl ether; a surface active fatty acid, such as palmitic acid or oleic acid; fatty acids; fatty acid monoglycerides; fatty acid diglycerides; fatty acid amides; sorbitan trioleate (Span®85) glycocholate; sorbitan monolaurate (Span®20); polysorbate 20 (Tween®20); polysorbate 60 (Tween®60); polysorbate 65 (Tween®65); polysorbate 80 (Tween®80); polysorbate 85 (Tween®85); polyoxyethylene monostearate; surfactin; a poloxomer; a sorbitan fatty acid ester such as sorbitan trioleate; lecithin; lysolecithin; phosphatidylserine; phosphatidylinositol;sphingomyelin; phosphatidylethanolamine (cephalin); cardiolipin; phosphatidic acid; cerebrosides; dicetylphosphate; dipalmitoylphosphatidylglycerol; stearylamine; dodecylamine; hexadecyl-amine; acetyl palmitate; glycerol ricinoleate; hexadecyl sterate; isopropyl myristate; tyloxapol; poly(ethylene glycol)5000-phosphatidylethanolamine; poly(ethylene glycol)400-monostearate; phospholipids; synthetic and/or natural detergents having high surfactant properties; deoxycholates; cyclodextrins; chaotropic salts; ion pairing agents; and combinations thereof. An amphiphilic entity component may be a mixture of different amphiphilic entities. Those skilled in the art will recognize that this is an exemplary, not comprehensive, list of substances with surfactant activity. Any amphiphilic entity may be used in the production of synthetic nanocarriers to be used in accordance with the present invention.

In some embodiments, synthetic nanocarriers may optionally comprise one or more carbohydrates. Carbohydrates may be natural or synthetic. A carbohydrate may be a derivatized natural carbohydrate. In certain embodiments, a carbohydrate comprises monosaccharide or disaccharide, including but not limited to glucose, fructose, galactose, ribose, lactose, sucrose, maltose, trehalose, cellbiose, mannose, xylose, arabinose, glucoronic acid, galactoronic acid, mannuronic acid, glucosamine, galatosamine, and neuramic acid. In certain embodiments, a carbohydrate is a polysaccharide, including but not limited to pullulan, cellulose, microcrystalline cellulose, hydroxypropyl methylcellulose (HPMC), hydroxycellulose (HC), methylcellulose (MC), dextran, cyclodextran, glycogen, hydroxyethylstarch, carageenan, glycon, amylose, chitosan, N,O-carboxylmethylchitosan, algin and alginic acid, starch, chitin, inulin, konjac, glucommannan, pustulan, heparin, hyaluronic acid, curdlan, and xanthan. In embodiments, the synthetic nanocarriers do not comprise (or specifically exclude) carbohydrates, such as a polysaccharide. In certain embodiments, the carbohydrate may comprise a carbohydrate derivative such as a sugar alcohol, including but not limited to mannitol, sorbitol, xylitol, erythritol, maltitol, and lactitol.

In some embodiments, synthetic nanocarriers can comprise one or more polymers. In some embodiments, the synthetic nanocarriers comprise one or more polymers that is a non-methoxy-terminated, pluronic polymer. In some embodiments, at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 97%, or 99% (weight/weight) of the polymers that make up the synthetic nanocarriers are non-methoxy-terminated, pluronic polymers. In some embodiments, all of the polymers that make up the synthetic nanocarriers are non-methoxy-terminated, pluronic polymers. In some embodiments, the synthetic nanocarriers comprise one or more polymers that is a non-methoxy-terminated polymer. In some embodiments, at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 97%, or 99% (weight/weight) of the polymers that make up the synthetic nanocarriers are non-methoxy-terminated polymers. In some embodiments, all of the polymers that make up the synthetic nanocarriers are non-methoxy-terminated polymers. In some embodiments, the synthetic nanocarriers comprise one or more polymers that do not comprise pluronic polymer. In some embodiments, at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 97%, or 99% (weight/weight) of the polymers that make up the synthetic nanocarriers do not comprise pluronic polymer. In some embodiments, all of the polymers that make up the synthetic nanocarriers do not comprise pluronic polymer. In some embodiments, such a polymer can be surrounded by a coating layer (e.g., liposome, lipid monolayer, micelle, etc.). In some embodiments, various elements of the synthetic nanocarriers can be attached to the polymer.

The immunosuppressants can be attached to the synthetic nanocarriers by any of a number of methods. Generally, the attaching can be a result of bonding between the immunosuppressants and the synthetic nanocarriers. This bonding can result in the immunosuppressants being attached to the surface of the synthetic nanocarriers and/or contained (encapsulated) within the synthetic nanocarriers. In some embodiments, however, the immunosuppressants are encapsulated by the synthetic nanocarriers as a result of the structure of the synthetic nanocarriers rather than bonding to the synthetic nanocarriers. In preferable embodiments, the synthetic nanocarrier comprises a polymer as provided herein, and the immunosuppressants are attached to the polymer.

When attaching occurs as a result of bonding between the immunosuppressants and synthetic nanocarriers, the attaching may occur via a coupling moiety. A coupling moiety can be any moiety through which an immunosuppressant is bonded to a synthetic nanocarrier. Such moieties include covalent bonds, such as an amide bond or ester bond, as well as separate molecules that bond (covalently or non-covalently) the immunosuppressant to the synthetic nanocarrier. Such molecules include linkers or polymers or a unit thereof. For example, the coupling moiety can comprise a charged polymer to which an immunosuppressant electrostatically binds. As another example, the coupling moiety can comprise a polymer or unit thereof to which it is covalently bonded.

In preferred embodiments, the synthetic nanocarriers comprise a polymer as provided herein. These synthetic nanocarriers can be completely polymeric or they can be a mix of polymers and other materials.

In some embodiments, the polymers of a synthetic nanocarrier associate to form a polymeric matrix. In some of these embodiments, a component, such as an immunosuppressant, can be covalently associated with one or more polymers of the polymeric matrix. In some embodiments, covalent association is mediated by a linker. In some embodiments, a component can be noncovalently associated with one or more polymers of the polymeric matrix. For example, in some embodiments, a component can be encapsulated within, surrounded by, and/or dispersed throughout a polymeric matrix. Alternatively or additionally, a component can be associated with one or more polymers of a polymeric matrix by hydrophobic interactions, charge interactions, van der Waals forces, etc. A wide variety of polymers and methods for forming polymeric matrices therefrom are known conventionally.

Polymers may be natural or unnatural (synthetic) polymers. Polymers may be homopolymers or copolymers comprising two or more monomers. In terms of sequence, copolymers may be random, block, or comprise a combination of random and block sequences. Typically, polymers in accordance with the present invention are organic polymers.

In some embodiments, the polymer comprises a polyester, polycarbonate, polyamide, or polyether, or unit thereof. In other embodiments, the polymer comprises poly(ethylene glycol) (PEG), polypropylene glycol, poly(lactic acid), poly(glycolic acid), poly(lactic-co-glycolic acid), or a polycaprolactone, or unit thereof. In some embodiments, it is preferred that the polymer is biodegradable. Therefore, in these embodiments, it is preferred that if the polymer comprises a polyether, such as poly(ethylene glycol) or polypropylene glycol or unit thereof, the polymer comprises a block-co-polymer of a polyether and a biodegradable polymer such that the polymer is biodegradable. In other embodiments, the polymer does not solely comprise a polyether or unit thereof, such as poly(ethylene glycol) or polypropylene glycol or unit thereof.

Other examples of polymers suitable for use in the present invention include, but are not limited to polyethylenes, polycarbonates (e.g. poly(1,3-dioxan-2one)), polyanhydrides (e.g. poly(sebacic anhydride)), polypropylfumerates, polyamides (e.g. polycaprolactam), polyacetals, polyethers, polyesters (e.g., polylactide, polyglycolide, polylactide-co-glycolide, polycaprolactone, polyhydroxyacid (e.g. poly(P-hydroxyalkanoate))), poly(orthoesters), polycyanoacrylates, polyvinyl alcohols, polyurethanes, polyphosphazenes, polyacrylates, polymethacrylates, polyureas, polystyrenes, and polyamines, polylysine, polylysine-PEG copolymers, and poly(ethyleneimine), poly(ethylene imine)-PEG copolymers.

In some embodiments, polymers in accordance with the present invention include polymers which have been approved for use in humans by the U.S. Food and Drug Administration (FDA) under 21 C.F.R. § 177.2600, including but not limited to polyesters (e.g., polylactic acid, poly(lactic-co-glycolic acid), polycaprolactone, polyvalerolactone, poly(1,3-dioxan-2one)); polyanhydrides (e.g., poly(sebacic anhydride)); polyethers (e.g., polyethylene glycol); polyurethanes; polymethacrylates; polyacrylates; and polycyanoacrylates.

In some embodiments, polymers can be hydrophilic. For example, polymers may comprise anionic groups (e.g., phosphate group, sulphate group, carboxylate group); cationic groups (e.g., quaternary amine group); or polar groups (e.g., hydroxyl group, thiol group, amine group). In some embodiments, a synthetic nanocarrier comprising a hydrophilic polymeric matrix generates a hydrophilic environment within the synthetic nanocarrier. In some embodiments, polymers can be hydrophobic. In some embodiments, a synthetic nanocarrier comprising a hydrophobic polymeric matrix generates a hydrophobic environment within the synthetic nanocarrier. Selection of the hydrophilicity or hydrophobicity of the polymer may have an impact on the nature of materials that are incorporated (e.g., attached) within the synthetic nanocarrier.

In some embodiments, polymers may be modified with one or more moieties and/or functional groups. A variety of moieties or functional groups can be used in accordance with the present invention. In some embodiments, polymers may be modified with polyethylene glycol (PEG), with a carbohydrate, and/or with acyclic polyacetals derived from polysaccharides (Papisov, 2001, ACS Symposium Series, 786:301). Certain embodiments may be made using the general teachings of U.S. Pat. No. 5543158 to Gref et al., or WO publication WO2009/051837 by Von Andrian et al.

In some embodiments, polymers may be modified with a lipid or fatty acid group. In some embodiments, a fatty acid group may be one or more of butyric, caproic, caprylic, capric, lauric, myristic, palmitic, stearic, arachidic, behenic, or lignoceric acid. In some embodiments, a fatty acid group may be one or more of palmitoleic, oleic, vaccenic, linoleic, alpha-linoleic, gamma-linoleic, arachidonic, gadoleic, arachidonic, eicosapentaenoic, docosahexaenoic, or erucic acid.

In some embodiments, polymers may be polyesters, including copolymers comprising lactic acid and glycolic acid units, such as poly(lactic acid-co-glycolic acid) and poly(lactide-co-glycolide), collectively referred to herein as “PLGA”; and homopolymers comprising glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA.” In some embodiments, exemplary polyesters include, for example, polyhydroxyacids; PEG copolymers and copolymers of lactide and glycolide (e.g., PLA-PEG copolymers, PGA-PEG copolymers, PLGA-PEG copolymers, and derivatives thereof. In some embodiments, polyesters include, for example, poly(caprolactone), poly(caprolactone)-PEG copolymers, poly(L-lactide-co-L-lysine), poly(serine ester), poly(4-hydroxy-L-proline ester), poly[α-(4-aminobutyl)-L-glycolic acid], and derivatives thereof.

In some embodiments, a polymer may be PLGA. PLGA is a biocompatible and biodegradable co-polymer of lactic acid and glycolic acid, and various forms of PLGA are characterized by the ratio of lactic acid:glycolic acid. Lactic acid can be L-lactic acid, D-lactic acid, or D,L-lactic acid. The degradation rate of PLGA can be adjusted by altering the lactic acid:glycolic acid ratio. In some embodiments, PLGA to be used in accordance with the present invention is characterized by a lactic acid:glycolic acid ratio of approximately 85:15, approximately 75:25, approximately 60:40, approximately 50:50, approximately 40:60, approximately 25:75, or approximately 15:85.

In some embodiments, polymers may be one or more acrylic polymers. In certain embodiments, acrylic polymers include, for example, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, polycyanoacrylates, and combinations comprising one or more of the foregoing polymers. The acrylic polymer may comprise fully-polymerized copolymers of acrylic and methacrylic acid esters with a low content of quaternary ammonium groups.

In some embodiments, polymers can be cationic polymers. In general, cationic polymers are able to condense and/or protect negatively charged strands of nucleic acids. Amine-containing polymers such as poly(lysine) (Zauner et al., 1998, Adv. Drug Del. Rev., 30:97; and Kabanov et al., 1995, Bioconjugate Chem., 6:7), poly(ethylene imine) (PEI; Boussif et al., 1995, Proc. Natl. Acad. Sci., USA, 1995, 92:7297), and poly(amidoamine) dendrimers (Kukowska-Latallo et al., 1996, Proc. Natl. Acad. Sci., USA, 93:4897; Tang et al., 1996, Bioconjugate Chem., 7:703; and Haensler et al., 1993, Bioconjugate Chem., 4:372) are positively-charged at physiological pH, form ion pairs with nucleic acids. In embodiments, the synthetic nanocarriers may not comprise (or may exclude) cationic polymers.

In some embodiments, polymers can be degradable polyesters bearing cationic side chains (Putnam et al., 1999, Macromolecules, 32:3658; Barrera et al., 1993, J. Am. Chem. Soc., 115:11010; Kwon et al., 1989, Macromolecules, 22:3250; Lim et al., 1999, J. Am. Chem. Soc., 121:5633; and Zhou et al., 1990, Macromolecules, 23:3399). Examples of these polyesters include poly(L-lactide-co-L-lysine) (Barrera et al., 1993, J. Am. Chem. Soc., 115:11010), poly(serine ester) (Zhou et al., 1990, Macromolecules, 23:3399), poly(4-hydroxy-L-proline ester) (Putnam et al., 1999, Macromolecules, 32:3658; and Lim et al., 1999, J. Am. Chem. Soc., 121:5633), and poly(4-hydroxy-L-proline ester) (Putnam et al., 1999, Macromolecules, 32:3658; and Lim et al., 1999, J. Am. Chem. Soc., 121:5633).

The properties of these and other polymers and methods for preparing them are well known in the art (see, for example, U.S. Pat. 6,123,727; 5,804,178; 5,770,417; 5,736,372; 5,716,404; 6,095,148; 5,837,752; 5,902,599; 5,696,175; 5,514,378; 5,512,600; 5,399,665; 5,019,379; 5,010,167; 4,806,621; 4,638,045; and 4,946,929; Wang et al., 2001, J. Am. Chem. Soc., 123:9480; Lim et al., 2001, J. Am. Chem. Soc., 123:2460; Langer, 2000, Acc. Chem. Res., 33:94; Langer, 1999, J. Control. Release, 62:7; and Uhrich et al., 1999, Chem. Rev., 99:3181). More generally, a variety of methods for synthesizing certain suitable polymers are described in Concise Encyclopedia of Polymer Science and Polymeric Amines and Ammonium Salts, Ed. by Goethals, Pergamon Press, 1980; Principles of Polymerization by Odian, John Wiley & Sons, Fourth Edition, 2004; Contemporary Polymer Chemistry by Allcock et al., Prentice-Hall, 1981; Deming et al., 1997, Nature, 390:386; and in U.S. Pat. 6,506,577, 6,632,922, 6,686,446, and 6,818,732.

In some embodiments, polymers can be linear or branched polymers. In some embodiments, polymers can be dendrimers. In some embodiments, polymers can be substantially cross-linked to one another. In some embodiments, polymers can be substantially free of cross-links. In some embodiments, polymers can be used in accordance with the present invention without undergoing a cross-linking step. It is further to be understood that the synthetic nanocarriers may comprise block copolymers, graft copolymers, blends, mixtures, and/or adducts of any of the foregoing and other polymers. Those skilled in the art will recognize that the polymers listed herein represent an exemplary, not comprehensive, list of polymers that can be of use in accordance with the present invention.

In some embodiments, synthetic nanocarriers do not comprise a polymeric component. In some embodiments, synthetic nanocarriers may comprise metal particles, quantum dots, ceramic particles, etc. In some embodiments, a non-polymeric synthetic nanocarrier is an aggregate of non-polymeric components, such as an aggregate of metal atoms (e.g., gold atoms).

The doses or dosage forms according to the invention can comprise pharmaceutically acceptable excipients, such as preservatives, buffers, saline, or phosphate buffered saline. The compositions may be made using conventional pharmaceutical manufacturing and compounding techniques to arrive at useful dosage forms. In some embodiments, the compositions of the dosings are suspended in sterile saline solution for injection together with a preservative. In some embodiments, synthetic nanocarriers are suspended in sterile saline solution for injection together with a preservative.

In embodiments, when preparing synthetic nanocarriers for use with immunosuppressants, methods for attaching components to the synthetic nanocarriers may be useful. If the component is a small molecule it may be of advantage to attach the component to a polymer prior to the assembly of the synthetic nanocarriers. In embodiments, it may also be an advantage to prepare the synthetic nanocarriers with surface groups that are used to attach the component to the synthetic nanocarrier through the use of these surface groups rather than attaching the component to a polymer and then using this polymer conjugate in the construction of synthetic nanocarriers.

In certain embodiments, the attaching can be with a covalent linker. In embodiments, components according to the invention can be covalently attached to the external surface via a 1,2,3-triazole linker formed by the 1,3-dipolar cycloaddition reaction of azido groups on the surface of the nanocarrier with a component containing an alkyne group or by the 1,3-dipolar cycloaddition reaction of alkynes on the surface of the nanocarrier with a component containing an azido group. Such cycloaddition reactions are preferably performed in the presence of a Cu(I) catalyst along with a suitable Cu(I)-ligand and a reducing agent to reduce Cu(II) compound to catalytic active Cu(I) compound. This Cu(I)-catalyzed azide-alkyne cycloaddition (CuAAC) can also be referred as the click reaction.

Additionally, the covalent attaching may comprise a covalent linker that comprises an amide linker, a disulfide linker, a thioether linker, a hydrazone linker, a hydrazide linker, an imine or oxime linker, an urea or thiourea linker, an amidine linker, an amine linker, and a sulfonamide linker.

An amide linker is formed via an amide bond between an amine on one component such as an immunosuppressant with the carboxylic acid group of a second component such as the nanocarrier. The amide bond in the linker can be made using any of the conventional amide bond forming reactions with suitably protected amino acids and activated carboxylic acid such N-hydroxysuccinimide-activated ester.

A disulfide linker is made via the formation of a disulfide (S—S) bond between two sulfur atoms of the form, for instance, of R1—S—S—R2. A disulfide bond can be formed by thiol exchange of a component containing thiol/mercaptan group(-SH) with another activated thiol group on a polymer or nanocarrier or a nanocarrier containing thiol/mercaptan groups with a component containing activated thiol group.

A triazole linker, specifically a 1,2,3-triazole of the form

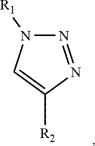

wherein R1 and R2 may be any chemical entities, is made by the 1,3-dipolar cycloaddition reaction of an azide attached to a first component, such as the nanocarrier, with a terminal alkyne attached to a second component, such as the immunosuppressant. The 1,3-dipolar cycloaddition reaction is performed with or without a catalyst, preferably with Cu(I)-catalyst, which links the two components through a 1,2,3-triazole function. This chemistry is described in detail by Sharpless et al., Angew. Chem. Int. Ed. 41(14), 2596, (2002) and Meldal, et al, Chem. Rev., 2008, 108(8), 2952-3015 and is often referred to as a “click” reaction or CuAAC.

In embodiments, a polymer containing an azide or alkyne group, terminal to the polymer chain is prepared. This polymer is then used to prepare a synthetic nanocarrier in such a manner that a plurality of the alkyne or azide groups are positioned on the surface of that nanocarrier. Alternatively, the synthetic nanocarrier can be prepared by another route, and subsequently functionalized with alkyne or azide groups. The component is prepared with the presence of either an alkyne (if the polymer contains an azide) or an azide (if the polymer contains an alkyne) group. The component is then allowed to react with the nanocarrier via the 1,3-dipolar cycloaddition reaction with or without a catalyst which covalently attaches the component to the particle through the 1,4-disubstituted 1,2,3-triazole linker.

A thioether linker is made by the formation of a sulfur-carbon (thioether) bond in the form, for instance, of R1—S—R2. Thioether can be made by either alkylation of a thiol/mercaptan (-SH) group on one component with an alkylating group such as halide or epoxide on a second component. Thioether linkers can also be formed by Michael addition of a thiol/mercaptan group on one component to an electron-deficient alkene group on a second component containing a maleimide group or vinyl sulfone group as the Michael acceptor. In another way, thioether linkers can be prepared by the radical thiol-ene reaction of a thiol/mercaptan group on one component with an alkene group on a second component.

A hydrazone linker is made by the reaction of a hydrazide group on one component with an aldehyde/ketone group on the second component.

A hydrazide linker is formed by the reaction of a hydrazine group on one component with a carboxylic acid group on the second component. Such reaction is generally performed using chemistry similar to the formation of amide bond where the carboxylic acid is activated with an activating reagent.

An imine or oxime linker is formed by the reaction of an amine or N-alkoxyamine (or aminooxy) group on one component with an aldehyde or ketone group on the second component.

An urea or thiourea linker is prepared by the reaction of an amine group on one component with an isocyanate or thioisocyanate group on the second component.

An amidine linker is prepared by the reaction of an amine group on one component with an imidoester group on the second component.

An amine linker is made by the alkylation reaction of an amine group on one component with an alkylating group such as halide, epoxide, or sulfonate ester group on the second component. Alternatively, an amine linker can also be made by reductive amination of an amine group on one component with an aldehyde or ketone group on the second component with a suitable reducing reagent such as sodium cyanoborohydride or sodium triacetoxyborohydride.

A sulfonamide linker is made by the reaction of an amine group on one component with a sulfonyl halide (such as sulfonyl chloride) group on the second component.

A sulfone linker is made by Michael addition of a nucleophile to a vinyl sulfone. Either the vinyl sulfone or the nucleophile may be on the surface of the nanocarrier or attached to a component.

The component, preferably an immunosuppressant, can also be conjugated to the nanocarrier via non-covalent conjugation methods. For example, a negative charged immunosuppressant can be conjugated to a positive charged nanocarrier through electrostatic adsorption. A component containing a metal ligand can also be conjugated to a nanocarrier containing a metal complex via a metal-ligand complex.

In embodiments, the component can be attached to a polymer, for example polylactic acid-block-polyethylene glycol, prior to the assembly of the synthetic nanocarrier or the synthetic nanocarrier can be formed with reactive or activatible groups on its surface. In the latter case, the component may be prepared with a group which is compatible with the attachment chemistry that is presented by the synthetic nanocarriers’ surface. In other embodiments, a peptide component can be attached to VLPs or liposomes using a suitable linker. A linker is a compound or reagent that is capable of attaching two molecules together. In an embodiment, the linker can be a homobifuntional or heterobifunctional reagent as described in Hermanson 2008. For example, an VLP or liposome synthetic nanocarrier containing a carboxylic group on the surface can be treated with a homobifunctional linker, adipic dihydrazide (ADH), in the presence of EDC to form the corresponding synthetic nanocarrier with the ADH linker. The resulting ADH linked synthetic nanocarrier is then conjugated with a peptide component containing an acid group via the other end of the ADH linker on nanocarrier to produce the corresponding VLP or liposome peptide conjugate.

For detailed descriptions of available conjugation methods, see Hermanson G T “Bioconjugate Techniques”, 2nd Edition Published by Academic Press, Inc., 2008. In addition to covalent attachment the component can be attached by adsorption to a pre-formed synthetic nanocarrier or it can be attached by encapsulation during the formation of the synthetic nanocarrier.

Any immunosuppressant as provided herein can be used and attached to the synthetic nanocarriers. Immunosuppressants include, but are not limited to, statins; mTOR inhibitors, such as rapamycin or a rapamycin analog; TGF-β signaling agents; TGF-β receptor agonists; histone deacetylase (HDAC) inhibitors; corticosteroids; inhibitors of mitochondrial function, such as rotenone; P38 inhibitors; NF-kβ inhibitors; adenosine receptor agonists; prostaglandin E2 agonists; phosphodiesterase inhibitors, such as phosphodiesterase 4 inhibitor; proteasome inhibitors; kinase inhibitors; G-protein coupled receptor agonists; G-protein coupled receptor antagonists; glucocorticoids; retinoids; cytokine inhibitors; cytokine receptor inhibitors; cytokine receptor activators; peroxisome proliferator-activated receptor antagonists; peroxisome proliferator-activated receptor agonists; histone deacetylase inhibitors; calcineurin inhibitors; phosphatase inhibitors and oxidized ATPs. Immunosuppressants also include IDO, vitamin D3, cyclosporine A, aryl hydrocarbon receptor inhibitors, resveratrol, azathiopurine, 6-mercaptopurine, aspirin, niflumic acid, estriol, tripolide, interleukins (e.g., IL-1, IL-10), cyclosporine A, siRNAs targeting cytokines or cytokine receptors and the like.

Examples of statins include atorvastatin (LIPITOR®, TORVAST®), cerivastatin, fluvastatin (LESCOL®, LESCOL® XL), lovastatin (MEVACOR®, ALTOCOR®, ALTOPREV®), mevastatin (COMPACTIN®), pitavastatin (LIVALO®, PIAVA®), rosuvastatin (PRAVACHOL®, SELEKTINE®, LIPOSTAT®), rosuvastatin (CRESTOR®), and simvastatin (ZOCOR®, LIPEX®).

Examples of mTOR inhibitors include rapamycin and analogs thereof (e.g., CCL-779, RAD001, AP23573, C20-methallylrapamycin (C20-Marap), C16-(S)-butylsulfonamidorapamycin (C16-BSrap), C16-(S)-3-methylindolerapamycin (C16-iRap) (Bayle et al. Chemistry & Biology 2006, 13:99-107)), AZD8055, BEZ235 (NVP-BEZ235), chrysophanic acid (chrysophanol), deforolimus (MK-8669), everolimus (RAD0001), KU-0063794, PI-103, PP242, temsirolimus, and WYE-354 (available from Selleck, Houston, TX, USA).

Examples of TGF-β signaling agents include TGF-β ligands (e.g., activin A, GDF1, GDF11, bone morphogenic proteins, nodal, TGF-βs) and their receptors (e.g., ACVR1B, ACVR1C, ACVR2A, ACVR2B, BMPR2, BMPR1A, BMPR1B, TGFβRI, TGFβRII), R-SMADS/co-SMADS (e.g., SMAD1, SMAD2, SMAD3, SMAD4, SMAD5, SMAD8), and ligand inhibitors (e.g., follistatin, noggin, chordin, DAN, lefty, LTBP1, THBS1, Decorin).

Examples of inhibitors of mitochondrial function include atractyloside (dipotassium salt), bongkrekic acid (triammonium salt), carbonyl cyanide m-chlorophenylhydrazone, carboxyatractyloside (e.g., from Atractylis gummifera), CGP-37157, (-)-Deguelin (e.g., from Mundulea sericea), F16, hexokinase II VDAC binding domain peptide, oligomycin, rotenone, Ru360, SFK1, and valinomycin (e.g., from Streptomyces fulvissimus) (EMD4Biosciences, USA).

Examples of P38 inhibitors include SB-203580 (4-(4-Fluorophenyl)-2-(4-methylsulfinylphenyl)-5-(4-pyridyl)1H-imidazole), SB-239063 (trans-1-(4hydroxycyclohexyl)-4-(fluorophenyl)-5-(2-methoxy-pyrimidin-4-yl) imidazole), SB-220025 (5-(2amino-4-pyrimidinyl)-4-(4-fluorophenyl)-1-(4-piperidinyl)imidazole)), and ARRY-797.

Examples of NF (e.g., NK-kβ) inhibitors include IFRD1, 2-(1,8-naphthyridin-2-yl)-Phenol, 5-aminosalicylic acid, BAY 11-7082, BAY 11-7085, CAPE (Caffeic Acid Phenethylester), diethylmaleate, IKK-2 Inhibitor IV, IMD 0354, lactacystin, MG-132 [Z-Leu-Leu-Leu-CHO], NFkB Activation Inhibitor III, NF-kB Activation Inhibitor II, JSH-23, parthenolide, Phenylarsine Oxide (PAO), PPM-18, pyrrolidinedithiocarbamic acid ammonium salt, QNZ, RO 106-9920, rocaglamide, rocaglamide AL, rocaglamide C, rocaglamide I, rocaglamide J, rocaglaol, (R)-MG-132, sodium salicylate, triptolide (PG490), and wedelolactone.

Examples of adenosine receptor agonists include CGS-21680 and ATL-146e.

Examples of prostaglandin E2 agonists include E-Prostanoid 2 and E-Prostanoid 4.

Examples of phosphodiesterase inhibitors (non-selective and selective inhibitors) include caffeine, aminophylline, IBMX (3-isobutyl-1-methylxanthine), paraxanthine, pentoxifylline, theobromine, theophylline, methylated xanthines, vinpocetine, EHNA (erythro-9-(2-hydroxy-3-nonyl)adenine), anagrelide, enoximone (PERFAN™), milrinone, levosimendon, mesembrine, ibudilast, piclamilast, luteolin, drotaverine, roflumilast (DAXAS™, DALIRESP™), sildenafil (REVATION®, VIAGRA®), tadalafil (ADCIRCA®, CIALIS®), vardenafil (LEVITRA®, STAXYN®), udenafil, avanafil, icariin, 4-methylpiperazine, and pyrazolo pyrimidin-7-1.

Examples of proteasome inhibitors include bortezomib, disulfiram, epigallocatechin-3-gallate, and salinosporamide A.

Examples of kinase inhibitors include bevacizumab, BIBW 2992, cetuximab (ERBITUX®), imatinib (GLEEVEC®), trastuzumab (HERCEPTIN®), gefitinib (IRESSA®), ranibizumab (LUCENTIS®), pegaptanib, sorafenib, dasatinib, sunitinib, erlotinib, nilotinib, lapatinib, panitumumab, vandetanib, E7080, pazopanib, and mubritinib.

Examples of glucocorticoids include hydrocortisone (cortisol), cortisone acetate, prednisone, prednisolone, methylprednisolone, dexamethasone, betamethasone, triamcinolone, beclometasone, fludrocortisone acetate, deoxycorticosterone acetate (DOCA), and aldosterone.

Examples of retinoids include retinol, retinal, tretinoin (retinoic acid, RETIN-A®), isotretinoin (ACCUTANE®, AMNESTEEM®, CLARAVIS®, SOTRET®), alitretinoin (PANRETIN®), etretinate (TEGISON™) and its metabolite acitretin (SORIATANE®), tazarotene (TAZORAC®, AVAGE®, ZORAC®), bexarotene (TARGRETIN®), and adapalene (DIFFERIN®).

Examples of cytokine inhibitors include ILlra, IL1 receptor antagonist, IGFBP, TNF-BF, uromodulin, Alpha-2-Macroglobulin, Cyclosporin A, Pentamidine, and Pentoxifylline (PENTOPAK®, PENTOXIL®, TRENTAL®).

Examples of peroxisome proliferator-activated receptor antagonists include GW9662, PPARγ antagonist III, G335, and T0070907 (EMD4Biosciences, USA).

Examples of peroxisome proliferator-activated receptor agonists include pioglitazone, ciglitazone, clofibrate, GW1929, GW7647, L-165,041, LY 171883, PPARγ activator, Fmoc-Leu, troglitazone, and WY-14643 (EMD4Biosciences, USA).

Examples of histone deacetylase inhibitors include hydroxamic acids (or hydroxamates) such as trichostatin A, cyclic tetrapeptides (such as trapoxin B) and depsipeptides, benzamides, electrophilic ketones, aliphatic acid compounds such as phenylbutyrate and valproic acid, hydroxamic acids such as vorinostat (SAHA), belinostat (PXD101), LAQ824, and panobinostat (LBH589), benzamides such as entinostat (MS-275), CI994, and mocetinostat (MGCD0103), nicotinamide, derivatives of NAD, dihydrocoumarin, naphthopyranone, and 2-hydroxynaphaldehydes.

Examples of calcineurin inhibitors include cyclosporine, pimecrolimus, voclosporin, and tacrolimus.

Examples of phosphatase inhibitors include BN82002 hydrochloride, CP-91149, calyculin A, cantharidic acid, cantharidin, cypermethrin, ethyl-3,4-dephostatin, fostriecin sodium salt, MAZ51, methyl-3,4-dephostatin, NSC 95397, norcantharidin, okadaic acid ammonium salt from prorocentrum concavum, okadaic acid, okadaic acid potassium salt, okadaic acid sodium salt, phenylarsine oxide, various phosphatase inhibitor cocktails, protein phosphatase 1C, protein phosphatase 2A inhibitor protein, protein phosphatase 2A1, protein phosphatase 2A2, and sodium orthovanadate.

### D. Methods of Making and Using the Compositions and Related Methods

Viral vectors can be made with methods known to those of ordinary skill in the art or as otherwise described herein. For example, viral vectors can be constructed and/or purified using the methods set forth, for example, in U.S. Pat. No. 4,797,368 and Laughlin et al., Gene, 23, 65-73 (1983).

Viral vectors, such as AAV vectors, may be produced using recombinant methods. For example, the methods can involve culturing a host cell which contains a nucleic acid sequence encoding an AAV capsid protein or fragment thereof; a functional rep gene; a recombinant AAV vector composed of AAV inverted terminal repeats (ITRs) and a transgene; and sufficient helper functions to permit packaging of the recombinant AAV vector into the AAV capsid proteins. The components to be cultured in the host cell to package a viral vector in a capsid may be provided to the host cell in trans. Alternatively, any one or more of the required components (e.g., recombinant viral vector, rep sequences, cap sequences, and/or helper functions) may be provided by a stable host cell which has been engineered to contain one or more of the required components using methods known to those of skill in the art. Most suitably, such a stable host cell can contain the required component(s) under the control of an inducible promoter. However, the required component(s) may be under the control of a constitutive promoter. The recombinant viral vector, rep sequences, cap sequences, and helper functions for producing the viral vector may be delivered to the packaging host cell using any appropriate genetic element. The selected genetic element may be delivered by any suitable method, including those described herein. The methods used to construct any embodiment of this invention are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present invention. See, e.g., K. Fisher et al, J. Virol., 70:520-532 (1993) and U.S. Pat. No. 5,478,745.

In some embodiments, recombinant AAV vectors may be produced using the triple transfection method (e.g., as described in detail in U.S. Pat. No. 6,001,650, the contents of which relating to the triple transfection method are incorporated herein by reference). Typically, the recombinant AAVs are produced by transfecting a host cell with a recombinant AAV vector (such as comprising a transgene) to be packaged into AAV particles, an AAV helper function vector, and an accessory function vector. Generally, an AAV helper function vector encodes AAV helper function sequences (rep and cap), which function in trans for productive AAV replication and encapsulation. Preferably, the AAV helper function vector supports efficient AAV vector production without generating any detectable wild-type AAV virions (i.e., AAV virions containing functional rep and cap genes). The accessory function vector can encode nucleotide sequences for non-AAV derived viral and/or cellular functions upon which AAV is dependent for replication. The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus. Other methods for producing viral vectors are known in the art. Moreover, viral vectors are available commercially.

In regard to synthetic nanocarriers attached to immunosuppressants, methods for attaching components to synthetic nanocarriers may be useful. Synthetic nanocarriers may be prepared using a wide variety of methods known in the art. For example, synthetic nanocarriers can be formed by methods such as nanoprecipitation, flow focusing using fluidic channels, spray drying, single and double emulsion solvent evaporation, solvent extraction, phase separation, milling, microemulsion procedures, microfabrication, nanofabrication, sacrificial layers, simple and complex coacervation, and other methods well known to those of ordinary skill in the art. Alternatively or additionally, aqueous and organic solvent syntheses for monodisperse semiconductor, conductive, magnetic, organic, and other nanomaterials have been described (Pellegrino et al., 2005, Small, 1:48; Murray et al., 2000, Ann. Rev. Mat. Sci., 30:545; and Trindade et al., 2001, Chem. Mat., 13:3843). Additional methods have been described in the literature (see, e.g., Doubrow, Ed., “Microcapsules and Nanoparticles in Medicine and Pharmacy,” CRC Press, Boca Raton, 1992; Mathiowitz et al., 1987, J. Control. Release, 5:13; Mathiowitz et al., 1987, Reactive Polymers, 6:275; and Mathiowitz et al., 1988, J. Appl. Polymer Sci., 35:755; U.S. Pat. 5578325 and 6007845; P. Paolicelli et al., “Surface-modified PLGA-based Nanoparticles that can Efficiently Associate and Deliver Virus-like Particles” Nanomedicine. 5(6):843-853 (2010)).

Various materials may be encapsulated into synthetic nanocarriers as desirable using a variety of methods including but not limited to C. Astete et al., “Synthesis and characterization of PLGA nanoparticles” J. Biomater. Sci. Polymer Edn, Vol. 17, No. 3, pp. 247-289 (2006); K. Avgoustakis “Pegylated Poly(Lactide) and Poly(Lactide-Co-Glycolide) Nanoparticles: Preparation, Properties and Possible Applications in Drug Delivery” Current Drug Delivery 1:321-333 (2004); C. Reis et al., “Nanoencapsulation I. Methods for preparation of drug-loaded polymeric nanoparticles” Nanomedicine 2:8- 21 (2006); P. Paolicelli et al., “Surface-modified PLGA-based Nanoparticles that can Efficiently Associate and Deliver Virus-like Particles” Nanomedicine. 5(6):843-853 (2010). Other methods suitable for encapsulating materials into synthetic nanocarriers may be used, including without limitation methods disclosed in U.S. Pat. 6,632,671 to Unger issued Oct. 14, 2003.

In certain embodiments, synthetic nanocarriers are prepared by a nanoprecipitation process or spray drying. Conditions used in preparing synthetic nanocarriers may be altered to yield particles of a desired size or property (e.g., hydrophobicity, hydrophilicity, external morphology, “stickiness,” shape, etc.). The method of preparing the synthetic nanocarriers and the conditions (e.g., solvent, temperature, concentration, air flow rate, etc.) used may depend on the materials to be attached to the synthetic nanocarriers and/or the composition of the polymer matrix.

If synthetic nanocarriers prepared by any of the above methods have a size range outside of the desired range, such synthetic nanocarriers can be sized, for example, using a sieve.

Elements (i.e., components) of the synthetic nanocarriers may be attached to the overall synthetic nanocarrier, e.g., by one or more covalent bonds, or may be attached by means of one or more linkers. Additional methods of functionalizing synthetic nanocarriers may be adapted from Published U.S. Pat. Application 2006/0002852 to Saltzman et al., Published U.S. Pat. Application 2009/0028910 to DeSimone et al., or Published International Patent Application WO/2008/127532 A1 to Murthy et al.

Alternatively or additionally, synthetic nanocarriers can be attached to components directly or indirectly via non-covalent interactions. In non-covalent embodiments, the non-covalent coupling is mediated by non-covalent interactions including but not limited to charge interactions, affinity interactions, metal coordination, physical adsorption, host-guest interactions, hydrophobic interactions, TT stacking interactions, hydrogen bonding interactions, van der Waals interactions, magnetic interactions, electrostatic interactions, dipole-dipole interactions, and/or combinations thereof. Such couplings may be arranged to be on an external surface or an internal surface of a synthetic nanocarrier. In embodiments, encapsulation and/or absorption is a form of coupling. In embodiments, the synthetic nanocarriers can be combined with a viral vector by admixing in the same vehicle or delivery system.

Compositions provided herein may comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol).

Compositions according to the invention may comprise pharmaceutically acceptable excipients. The compositions may be made using conventional pharmaceutical manufacturing and compounding techniques to arrive at useful dosage forms. Techniques suitable for use in practicing the present invention may be found in Handbook of Industrial Mixing: Science and Practice, Edited by Edward L. Paul, Victor A. Atiemo-Obeng, and Suzanne M. Kresta, 2004 John Wiley & Sons, Inc.; and Pharmaceutics: The Science of Dosage Form Design, 2nd Ed. Edited by M. E. Auten, 2001, Churchill Livingstone. In an embodiment, compositions are in a sterile saline solution for injection together with a preservative.

It is to be understood that the compositions of the invention can be made in any suitable manner, and the invention is in no way limited to compositions that can be produced using the methods described herein. Selection of an appropriate method of manufacture may require attention to the properties of the particular moieties being associated.

In some embodiments, compositions are manufactured under sterile conditions or are terminally sterilized. This can ensure that resulting compositions are sterile and non-infectious, thus improving safety when compared to non-sterile compositions. This provides a valuable safety measure, especially when subjects receiving the compositions have immune defects, are suffering from infection, and/or are susceptible to infection. In some embodiments, the compositions may be lyophilized and stored in suspension or as lyophilized powder depending on the formulation strategy for extended periods without losing activity.

Administration according to the present invention may be by a variety of routes, including but not limited to subcutaneous, intravenous, or intraperitoneal routes. The compositions referred to herein may be manufactured and prepared for administration, such as concomitant administration, using conventional methods.

The compositions of the invention can be administered in effective amounts, such as the effective amounts described elsewhere herein. Doses of dosage forms may contain varying amounts of immunosuppressants, according to the invention. Doses of dosage forms may contain varying amounts of viral vectors, according to the invention. The amount of respective components present in the dosage forms can be varied according to the nature of the components, the therapeutic benefit to be accomplished, and other such parameters. In embodiments, dose ranging studies can be conducted to establish optimal therapeutic amounts of the components to be present in the dosage forms. In embodiments, the components are present in the dosage forms in an amount effective to reduce an undesired humoral immune response to the viral vector and/or increased or durable expression upon administration to a subject. It may be possible to determine amounts of the components effective to reduce an undesired humoral immune response using conventional dose ranging studies and techniques in subjects. Dosage forms may be administered at a variety of frequencies (i.e., according to an administration schedule).

Another aspect of the disclosure relates to kits. In some embodiments, the kit comprises one or more first doses and one or more second doses and, optionally, one or more third doses, as provided herein. Each of the doses of a kit can be contained within separate containers or within the same container in the kit. In some embodiments, the container is a vial or an ampoule. In some embodiments, each of the doses can be contained within a solution separate from the container, such that the dose may be added to a container at a subsequent time. In some embodiments, the doses are in lyophilized form each in a separate container or in the same container, such that they may be reconstituted at a subsequent time. In some embodiments, the kit further comprises instructions for reconstitution, mixing, administration, etc. In some embodiments, the instructions include a description of the methods described herein. Instructions can be in any suitable form, e.g., as a printed insert or a label. In some embodiments, the kit further comprises one or more syringes.

An administration schedule can be determined by varying the number of dosing(s) and/or the length of time between the dosing(s) and assessing an undesired humoral immune response to a viral vector and/or expression of a transgene or nucleic acid material thereof. For example, after administering first dosing(s) and second dosing(s) and, optionally, third dosing(s) an undesired humoral immune response to a viral vector and/or expression can be measured. This undesired humoral immune response and/or expression can be compared to the same type of immune response and/or expression that occurs without the first and second dosing(s) and, optionally third dosing(s), such as when only one or more dosings of viral vector has occurred without concomitant administration with synthetic nanocarriers attached to an immunosuppressant or other dosing(s) as provided herein. Generally, if it is found that the level of the undesired immune response is reduced or expression is increased or persists for a certain period of time, an administration schedule can be beneficial for subjects in need of treatment with a viral vector and can be used with the methods and compositions of the invention provided herein. Administration schedules may be determined by starting with a test schedule and using known scaling techniques (such as allometric or isometric scaling) as appropriate. In another embodiment, the administration schedule may be determined by testing various schedules in a subject, e.g., through direct experimentation based on experience and guiding data.

### EXAMPLES

### Example 1: Synthesis of Synthetic Nanocarriers Comprising an Immunosuppressant

Synthetic nanocarriers comprising an immunosuppressant, for example rapamycin, were produced. Preferably, in some embodiments of any one of the methods or compositions provided herein the synthetic nanocarriers comprising an immunosuppressant are produced by any one of the methods of U.S. Publication No. US 2016/0128986 A1 and US Publication No. US 2016/0128987 A1, the described methods of such production and the resulting synthetic nanocarriers being incorporated herein by reference in their entirety. In any one of the methods or compositions provided herein, the synthetic nanocarriers comprising an immunosuppressant, such as rapamycin (e.g., encapsulated rapamycin), are such incorporated synthetic nanocarriers. In any one of the methods or compositions provided herein, the synthetic nanocarriers comprise polymers, such as PLA, PLGA or PCL and/or pegylated versions of such polymers. Exemplary synthetic nanocarriers are and may be referred to herein as IMMTOR.

### Example 2: Non-Human Primates Study, Multiple Benefits of Synthetic Nanocarriers Comprising Immunosuppressant in Viral Vector Therapy

It has been found that co-administration of AAV vector and synthetic nanocarriers comprising an immunosuppressant, for example rapamycin, in non-human primates (NHP) results in a significant first dose effect, inducing higher and more durable transgene expression as compared to administration of AAV vector alone. Also, robust inhibition of anti-AAV8 IgG and neutralizing antibodies were achieved when synthetic nanocarriers comprising an immunosuppressant, for example rapamycin, were administered with AAV vector, an effect that was strengthened by repeat dosing of the synthetic nanocarriers comprising the immunosuppressant, indicating the ability of the synthetic nanocarriers comprising the immunosuppressant to enable re-dosing of AAV gene therapies. Further, the data support the treatment of methylmalonic acidemia (MMA) and ornithine transcarbamylase (OTC) deficiency with gene therapy in combination the synthetic nanocarriers comprising an immunosuppressant, for example rapamycin. The data provided herein demonstrate the efficacy, safety and durability of adeno-associated viral (AAV) vector gene therapies with co-administration of an AAV vector and synthetic nanocarriers comprising an immunosuppressant, for example rapamycin, in non-human primates.

The finding that co-administration of AAV vector and synthetic nanocarriers comprising an immunosuppressant, for example rapamycin, leads to higher transgene expression demonstrates the ability to use lower levels of dosing of AAV gene therapies when combined with administration of synthetic nanocarriers comprising the immunosuppressant. This can improve patient safety and lower costs. Further, long-term gene therapy data demonstrate that expression of systemic AAV gene therapies may wane over time, a limitation that the synthetic nanocarriers comprising an immunosuppressant, for example rapamycin, can address. Finally, AAV gene therapies cannot be re-dosed without the synthetic nanocarriers comprising the immunosuppressant, for example rapamycin, due to the formation of neutralizing antibodies to the AAV vector. These data show that the synthetic nanocarriers comprising an immunosuppressant, for example rapamycin, mitigates the formation of these neutralizing antibodies in NHPs, thereby allowing for re-dosing. Thus, the compositions and methods for administration provided herein can allow for lower doses of a viral vector, such as an AAV vector, and/or can allow for incremental gene therapy redosing.

Specifically, the administration of a single intravenous (IV) infusion of a recombinant adeno-associated serotype eight capsid directing expression of a transgene encoding secreted embryonic alkaline phosphatase (AAV8-SEAP), a widely used reporter gene transgene, either alone or co-administered with synthetic nanocarriers comprising rapamycin were evaluated in NHP. Five cohorts of NHP each received 2×10¹² vector genomes (vg)/kilogram (kg) of AAV8-SEAP either alone or in combination with one of two dose levels of synthetic nanocarriers comprising rapamycin (3 or 6 mg/kg) at day 0. Cohort 3 received 6 mg/kg of the synthetic nanocarriers comprising rapamycin admixed with AAV8-SEAP prior to infusion. All other cohorts received sequential infusions of the synthetic nanocarriers comprising rapamycin followed by AAV8-SEAP. Cohorts four and five received additional doses of the synthetic nanocarriers comprising rapamycin at day 28 and day 56 of the study, with cohort five also receiving additional low doses of AAV8-SEAP (0.2×10¹² vg/kg) at day 28 and day 56.

Results include:
Transgene expression peaked at Day 28 in animals receiving AAV8-SEAP alone. At Day 28, cohorts treated with AAV8-SEAP + synthetic nanocarriers comprising rapamycin showed consistently higher levels of transgene expression, indicating a first dose benefit of the synthetic nanocarriers comprising rapamycin on transgene expression.
After Day 28 serum SEAP levels in the cohort treated with AAV8-SEAP alone dropped precipitously, whereas cohorts treated with AAV8-SEAP + synthetic nanocarriers comprising rapamycin showed stable expression of SEAP through Day 84, demonstrating the synthetic nanocarriers notable impact on durability of transgene expression. Cohort 5 that received two additional low doses of AAV8-SEAP on Days 28 and 56 showed an incremental trend in increased transgene expression at Days 56 and 84.
All synthetic nanocarrier-treated cohorts achieved robust inhibition of anti-AAV8 IgG antibodies through day 56. This effect was strengthened with repeat-dosing of the synthetic nanocarriers comprising rapamycin at days 28 and 56. Five out of six animals in the cohorts that received three monthly doses of the synthetic nanocarriers comprising rapamycin had neutralizing antibody titers of less than 1:5 at day 84, as measured with a cell-based neutralizing assay, while the sixth animal showed a low titer of 1:8. In contrast, all three animals treated with AAV8-SEAP alone had neutralizing antibody titers greater than 1:3400.
Overall, there was a high degree of correlation between Day 84 anti-AAV8 IgG and neutralizing antibody titers across all animals and all cohorts.

### Example 3: Repeated, Concomitant Administration With Lower Doses (Prophetic)

As provided herein, a clinician can select a dose of the viral vector. However, in light of the inventor’s findings, a clinician may now select and use lower doses of the viral vector when synthetic nanocarriers attached to an immunosuppressant is administered at least once concomitantly and, optionally, repeatedly. The lower dose is any amount lower than would have otherwise been selected for the subject. In an embodiment, the lower dose is lower but no less than ⅒ of the dose that would have been selected without the at least one concomitant administration of synthetic nanocarriers attached to an immunosuppressant as provided herein.

Accordingly, any one of the subjects provided herein can be treated with repeated, concomitant, such as simultaneous, administration of any one of the viral vectors provided herein and any one of the populations of synthetic nanocarriers attached to an immunosuppressant provided herein where the doses of the viral vector are selected to be less than the dose of the viral vector that would have been selected for the subject (for example, less than but at least ⅒ the dose) without the administration of the synthetic nanocarriers. Each dose of the viral vector of the repeated, concomitant administration may be less than (for example, less than but at least ⅒ the dose) what would have otherwise been selected.

### Example 4: Efficient Suppression of IgG Antibody Responses to High Doses of AAV8 Capsids by Single and Multiple Administrations of Synthetic Nanocarriers Attached to an Immunosuppressant

Achieving durable systemic AAV gene therapy may require repeat AAV dosing. Re-dosing may be prevented by the formation of neutralizing antibodies. It has been demonstrated that tolerogenic synthetic nanocarriers encapsulating rapamycin (e.g., ImmTOR) can mitigate AAV immunogenicity and enable vector redosing in mice and nonhuman primates at moderate vector doses of ~2e12 vg/kg. The ability of the nanocarriers to block IgG formation using higher doses of AAV8 empty capsids (AAV8-EC) was evaluated. A single dose of 100 µg of the nanocarriers (e.g., ImmTOR) completely abrogated IgG responses to 2e13 vector particles (vp)/kg AAV8-EC through day 62 in Balb/C mice and in the majority (10/12) of C57BL/6 mice at 2-6e12 vp/kg AAV8-EC. However, the nanocarriers (e.g., ImmTOR) were less efficient at 2e13 vp/kg in C57BL/6 mice, with delayed breakthrough of antibodies observed in most animals at 200 µg. Higher doses of the nanocarriers (300 µg) inhibited IgG formation in the majority of mice (9/12) at 2E13 vp/kg. It is thought that late IgG seroconversions may be a consequence of prolonged AAV circulation due to inhibition of the early anti-capsid immune response.

The administration of two additional monthly doses of the nanocarriers (e.g., ImmTOR) was also evaluated. Mice treated with 2E13 vp/kg capsid and three 200-300 µg monthly nanocarrier (e.g., ImmTOR) doses developed little or no IgG through Day 84. Thus, repeated administration of the nanocarriers can provide more durable suppression of antibodies against higher viral capsid doses.

### Example 5: Efficient Suppression of IgG Antibody Responses to High Doses of AAV8 Capsids by Single and Multiple Administrations of Synthetic Nanocarriers Attached to an Immunosuppressant

Achieving durable systemic AAV gene therapy may require repeat AAV dosing. Currently, re-dosing is prevented by the formation of neutralizing antibodies. It has been demonstrated that tolerogenic synthetic nanocarriers encapsulating rapamycin mitigate AAV immunogenicity and enable vector redosing in mice and nonhuman primates at moderate vector doses of ~2e¹² vg/kg. The ability of such nanocarriers to block IgG formation using higher doses of AAV8 empty capsids (AAV8-EC) has been evaluated. A single dose of 100 µg the nanocarriers completely abrogated IgG responses to 2e¹³ vector particles (vp)/kg AAV8-EC through day 62 in Balb/C mice and in the majority (10/12) of C57BL/6 mice at 2-6e¹² vp/kg AAV8-EC. However, the nanocarriers were less efficient at 2e¹³ vp/kg in C57BL/6 mice, with delayed breakthrough of antibodies observed in most animals at 200 µg. Higher doses of ImmTOR (300 µg) inhibited IgG formation in the majority of mice (9/12) at 2E¹³ vp/kg.

Administration of two additional monthly doses of the nanocarriers was also evaluated. Mice treated with 2E¹³ vp/kg capsid and three 200-300 µg monthly nanocarrier doses developed little or no IgG through Day 84. Thus, repeated administration of synthetic nanocarriers encapsulating rapamycin can provide more durable suppression of antibodies against higher viral capsid doses.

The results demonstrate that AAV8-EC empty capsids are moderately immunogenic in BALB/c and strongly immunogenic in C57BL/6 mice over the dose range tested (2×10¹²-2×10¹³ vg/kg). Complete IgG suppression by 100 µg single-dose synthetic nanocarriers to 2×10¹²-2×10¹³ vg/kg AAV8-EC in BALB/c mice was observed. Strong IgG suppression by 200 µg single-dose synthetic nanocarriers to 2-6×10¹² vg/kg AAV8-EC in C57BL/6 mice was also observed. Complete or near-complete long-term IgG suppression with three-monthly doses of synthetic nanocarriers even to a high dose of 2×10¹³ vg/kg AAV8-EC in C57BL/6 mice was further observed.

### Example 6: Enhanced Level and Durability of AAV Transgene Expression and Mitigation of Anti-capsid Neutralizing Antibodies by Tolerogenic Synthetic Nanocarriers Encapsulating Rapamycin in Nonhuman Primates

Immune responses to the capsid or transgene product can lead to the loss of transgene product and the formation of neutralizing anti-AAV8 antibodies (NAb), which prevent the ability to re-dose patients. Synthetic nanocarriers encapsulating rapamycin have been shown to selectively mitigate AAV immunogenicity and enable vector redosing. The impact of different dosing regimens of AAV8 encoding human secreted embryonic alkaline phosphatase (AAV8-SEAP) and such synthetic nanocarriers on NAb formation and SEAP activity in nonhuman primates (NHPs) was explored. As expected, the control group had an early anti-AAV8 IgM response that transitioned to an anti-AAV IgG response and strong NAb titers by day 84. SEAP activity peaked at day 28 and rapidly declined by day 84, suggestive of an anti-SEAP antibody response. In contrast, the addition of a single dose of the synthetic nanocarriers delayed anti-AAV8 IgG antibody formation until at least day 56 and reduced NAbs on day 84 in some animals. Treatment with the synthetic nanocarriers led to increased and sustained SEAP activity in comparison to the control group. The impact of the synthetic nanocarriers was most striking in groups with 3 monthly doses of the synthetic nanocarriers, in which anti-AAV8 IgM, IgG and neutralizing antibodies were mitigated. Five of 6 animals had NAb titers < 1:5 and the sixth animal had a weak titer of 1:11. Combined with the enhanced and sustained expression of SEAP in these animals, these results indicate that 3 monthly doses of the synthetic nanocarriers may enhance the level and durability of transgene expression, while inhibiting the formation of NAbs and enabling the possibility of vector re-administration.

Five groups of NHPs were administered AAV8-SEAP and/or synthetic nanocarriers encapsulating rapamycin intraveneously. When dosed with both AAV8-SEAP and the synthetic nanocarriers, animals received the treatments sequentially except for group 3, in which AAV8-SEAP and the synthetic nanocarriers were admixed together and dosed via a single infusion. Animals were bled on days 0, 7, 14, 28, 56 and 84 to assess serum SEAP activity, anti-AAV8 IgG and anti-AAV8 IgM levels. Neutralizing antibody levels were assessed on day 84. Serum SEAP expression was determined using the Phospha-Light™ SEAP Reporter Gene Assay System (Invitrogen, Carlsbad, CA). Anti-AAV8 IgG and IgM were determined using direct bind ELISAs. NAb antibodies were assessed using a HEK-293 AAV8-Luc cell-based assay.

While a single 6 mg/kg dose of synthetic nanocarriers (B, C) delays induction of anti-AAV8 IgG and IgM antibodies compared to the control group (A), by day 84 both groups show an increasing level of anti-AAV8 IgG antibodies. In contrast, in groups that received 3 monthly doses of synthetic nanocarriers (D,E), one animal that had pre-existing anti-AAV8 IgM was only transiently positive on day 7 and one animal was positive on day 84 for anti-AAV8 IgM. While anti-AAV8 IgG antibodies were observed transiently, with signals just above the assay cut point, only 1 animal had anti-AAV8 IgG antibodies on day 84 when treated with 3 monthly doses of the synthetic nanocarriers.

The results demonstrate that administration of the synthetic nanocarriers encapsulating rapamycin with AAV8-SEAP led to enhanced SEAP activity that was durable until the end of the study on day 84. While a single dose of the synthetic nanocarriers was able to delay the formation of anti-AAV8 antibodies, 3 monthly doses of the synthetic nanocarriers was optimal in mitigating the development anti-AAV8 IgG antibody and NAbs. Three monthly doses of synthetic nanocarriers can enhance the level and durability of transgene expression, while inhibiting the formation of NAbs and enabling the possibility of vector re-administration.

1. A method comprising:
(1) a first dosing that comprises concomitantly administering
(a) a viral vector, such as an AAV vector, that is not attached to any synthetic nanocarriers, and
(b) synthetic nanocarriers that are attached to an immunosuppressant, such as rapamycin, and that comprise no viral vector antigen-presenting cell (APC) presentable antigens of the viral vector;
(2) a second dosing that comprises concomitantly administering
(c) the synthetic nanocarriers that are attached to an immunosuppressant and that comprise no viral vector APC antigens of the viral vector and the viral vector; and
(3) administering the first and second dosings to a subject according to an administration schedule that reduces an undesired humoral immune response to the viral vector and/or increases transgene or nucleic acid material expression or provides durable transgene or nucleic acid material expression, such as for at least one month from the first or second dosing,
wherein the viral vector of the first and second dosings is at a higher dose such as at least 1e¹³ or 2e¹³ vg/kg.

2. The method of claim 1, wherein the method further comprises:
(4) a third dosing that comprises concomitantly administering
(d) the synthetic nanocarriers that are attached to an immunosuppressant and that comprise no viral vector APC antigens of the viral vector and the viral vector,
wherein the viral vector is also at a higher dose such as at least 1e¹³ or 2e¹³ vg/kg; and
(5) administering the third dosing to a subject also according to an administration schedule that reduces an undesired humoral immune response to the viral vector and/or increases transgene or nucleic acid material expression or provides durable transgene or nucleic acid material expression, such as for at least one month from the third dosing.

3. The method of claim 1, further comprising (6) determining the administration schedule for the first and second dosings or first, second and third dosings that reduces an undesired humoral immune response to the viral vector and/ or increases transgene or nucleic acid material expression or provides durable transgene or nucleic acid material expression, such as for at least one month from each first dosing.

4. The method of claim 1, wherein the higher dose is a therapeutically effective dose for a human.

5. A method comprising:
(1) a first dosing that comprises concomitantly administering
(a) a viral vector, such as an AAV vector, that is not attached to any synthetic nanocarriers, and
(b) synthetic nanocarriers that are attached to an immunosuppressant, such as rapamycin, and that comprise no viral vector antigen-presenting cell (APC) presentable antigens of the viral vector;
(2) a second dosing that comprises administering
(c) the synthetic nanocarriers that are attached to an immunosuppressant and that comprise no viral vector APC antigens of the viral vector and without concomitant administration of the viral vector or concomitantly the synthetic nanocarriers that are attached to an immunosuppressant and that comprise no viral vector APC antigens of the viral vector and the viral vector, wherein the viral vector is at a dose lower than the dose of the viral vector of the first dosing; and
(3) administering the first and second dosings to a subject according to an administration schedule that reduces an undesired humoral immune response to the viral vector and/or increases transgene or nucleic acid material expression or provides durable transgene or nucleic acid material expression, such as for at least one month or two months from the first dosing.

6. The method of claim 5, wherein the method further comprises:
(4) a third dosing that comprises administering
(d) the synthetic nanocarriers that are attached to an immunosuppressant and that comprise no viral vector APC antigens of the viral vector and without concomitant administration of the viral vector or concomitantly the synthetic nanocarriers that are attached to an immunosuppressant and that comprise no viral vector APC antigens of the viral vector and the viral vector, wherein the viral vector is at a dose lower than the dose of the viral vector of the first dosing; and
(5) administering the third dosing to a subject also according to an administration schedule that reduces an undesired humoral immune response to the viral vector and/or increases transgene or nucleic acid material expression or provides durable transgene or nucleic acid material expression, such as for at least one month, two months or three months from the first dosing.

7. The method of claim 5, further comprising (6) determining the administration schedule for the first and second dosings or first, second and third dosings that reduces an undesired humoral immune response to the viral vector and/ or increases transgene or nucleic acid material expression or provides durable transgene or nucleic acid material expression, such as for at least one month, two months or three months from the first dosing.

8. The method of claim 5, wherein the lower dose of the viral vector of the second or third dosings is less than but at least ⅒ of the dose of the viral vector of the first dosing.

9. The method of claim 1, wherein the second dosing is or is about a month after the first dosing.

10. The method of claim 1, wherein the third dosing is or is about a month after the second dosing.

11. The method of claim 1, wherein the method further comprises:
(a) assessing the undesired humoral immune response and/or transgene or nucleic acid material expression in the subject prior to and/or after the administration of the first dosing, second dosing and/or third dosing; or
(b) identifying the subject as having or at risk of having an undesired humoral immune response to the viral vector and/or as being in need of effective or durable transgene or nucleic acid material expression, such as for at least one month, two month or three months.

12-13. (canceled)

14. A composition comprising:
(1) one or more first doses that each comprise
(a) a viral vector, such as an AAV vector, that is not attached to any synthetic nanocarriers, and/or
(b) synthetic nanocarriers that are attached to an immunosuppressant, such as rapamycin, and that comprise no viral vector antigen-presenting cell (APC) presentable antigens of the viral vector,
wherein the one or more first doses in combination comprise (a) and (b); and
(2) one or more second doses and, optionally, one or more third doses that each comprise
(c) the synthetic nanocarriers that are attached to an immunosuppressant that comprise no viral vector APC presentable antigens of the viral vector and without a viral vector or (i) the synthetic nanocarriers that are attached to an immunosuppressant that comprise no viral vector APC presentable antigens of the viral vector and/or (ii) the viral vector, wherein the viral vector is at a dose of any one of the precedingclaims or lower than the one or more first doses, wherein the one or more second doses and/or one or more third doses in combination comprise (i) and (ii);
optionally, for use in a method of reducing an undesired humoral immune response to the viral vector and/or increasing transgene or nucleic acid material expression or providing durable transgene or nucleic acid material expression, wherein the method comprises administering the first and second doses and, optionally, third doses to a subject according to an administration schedule.

15. The composition of claim 14, wherein the method further comprises determining the administration schedule for the first and second doses and, optionally, third doses that reduces an undesired humoral immune response to the viral vector and/or increases transgene or nucleic acid material expression or provides durable transgene or nucleic acid material expression.

16-18. (canceled)

19. The method of claim 1, wherein the immunosuppressants comprise a statin, an mTOR inhibitor, a TGF-β signaling agent, a corticosteroid, an inhibitor of mitochondrial function, a P38 inhibitor, an NF-_{K}B inhibitor, an adenosine receptor agonist, a prostaglandin E2 agonist, a phosphodiesterase 4 inhibitor, an HDAC inhibitor or a proteasome inhibitor.

20-21. (canceled)

22. The method of claim 1, wherein the viral vector is an AAV vector, such as an AAV8 vector.

23. The method of claim 1, wherein the viral vector is for treating MMA or OTC.

24. The method of claim 1, wherein a load of the immunosuppressant is on average across the population of synthetic nanocarriers is between 0.1% and 50%.

25. (canceled)

26. The method of claim 1, wherein the synthetic nanocarriers are polymeric.

27-31. (canceled)

32. The method of claim 1, wherein the mean of a particle size distribution obtained using dynamic light scattering of the synthetic nanocarriers of the population is a diameter greater than 100 nm.

33-43. (canceled)

44. The method of claim 1, wherein an aspect ratio of the synthetic nanocarriers of the population is greater than or equal to 1:1, 1:1.2, 1:1.5, 1:2, 1:3, 1:5, 1:7 or 1:10.
